(19) United States
(12) Patent Application Publication
(10) Pub. No.: US 2024/0191288 A1
(43) Pub. Date: Jun. 13, 2024

(54) BLOCKING OLIGONUCLEOTIDES FOR THE SELECTIVE DEPLETION OF NON-DESIRABLE FRAGMENTS FROM AMPLIFIED LIBRARIES

(71) Applicant: Illumina, Inc., San Diego, CA (US)

(72) Inventors: Colin Brown, San Diego, CA (US); Sarah Shultzaberger, San Diego, CA (US); Stephen M. Gross, San Diego, CA (US); Angelica Barr, San Diego, CA (US); Samantha Snow, San Diego, CA (US)

(21) Appl. No.: 18/285,222

(22) PCT Filed: Mar. 30, 2022
(86) PCT No.: PCT/US2022/022663

§ 371 Date: Sep. 29, 2023

Related U.S. Application Data
(60) Provisional application No. 63/169,185, filed on Mar. 31, 2021.

(51) Int. Cl. C12Q 1/6848; C12Q 1/48; C12Q 1/6855

(52) U.S. Cl. CPC C12Q 1/6848; C12Q 1/485; C12Q 1/6855

(57) ABSTRACT

The disclosure relates to methods, compositions, and kits for the selective depletion of non-desirable fragments from amplified libraries using blocking oligonucleotides.

### CROSS REFERENCE TO RELATED APPLICATIONS

This application claims priority to U.S. Provisional Application Ser. No. 63/169,185, filed on Mar. 31, 2021, the disclosures of which are incorporated herein by reference.

### TECHNICAL FIELD

The disclosure relates to methods, compositions, and kits for the selective depletion of non-desirable fragments from amplified libraries using blocking oligonucleotides.

### BACKGROUND

Library preparation aims to build a collection of DNA fragments for next-generation sequencing (NGS). A high-quality DNA library guarantees uniform and consistent genome coverage, thus delivering comprehensive and reliable sequencing data. Library preparations, however, contain many non-desirable sequences, such as sequences for rRNA, sequences for housekeeping genes, mitochondrial sequences, etc. As such, the elimination of these non-desirable sequences in library preparations can provide more focused and data-rich Next Generation Sequencing (NGS) libraries.

### SUMMARY

Current methods for depletion of abundant sequences, such as hybridization pull-down of rRNA (e.g., RiboZero, RiboMinus) or enzymatic digestion (e.g., RNaseH, CRISPR) perform well for high-quality, high-input samples, but often show poor performance with lower-quality, less abundant inputs encountered in clinically-relevant sample types such as formalin fixed/paraffin-embedded (FFPE) tissue and plasma-derived circulating RNA (C-RNA). Alternatively, sequence-specific enrichment approaches (e.g., exome capture) show better performance for low-input samples, but are restricted by the need to pre-specify a set of targets. This limits their utility for detecting rare transcript isoforms and non-coding RNAs that may be useful biomarkers.

The disclosure provides an alternative depletion strategy, “PCR Blocking”, that uses long, strongly binding oligonucleotides to block polymerase extension in PCR and related methods. The approach described herein eliminates the time-consuming and inefficient incubation and purification steps characteristic of existing approaches, and is expected to improve library conversion in low-input applications by allowing abundant sequences to act as a built-in ‘carrier’ during steps prior to amplification.

In a particular embodiment, the disclosure provides a method to selectively deplete non-desirable fragments from amplified DNA or cDNA libraries by using one or more blocking oligonucleotides, comprising: amplifying in a polymerase chain reaction (PCR) reaction, a plurality of library fragments comprising a double stranded template sequence including adapter sequences, wherein a portion of the fragments comprise non-desirable fragments that are not to be analyzed; wherein the PCR reaction comprises a plurality of fragments, a polymerase, dNTPS, PCR primers, and one or more blocking oligonucleotides, wherein the one or more blocking oligonucleotides comprise (i) and/or (ii), and (iii): (i) at the 5′ terminus, one or more nucleotides that comprise a phosphorothioate linkage; and/or (ii) at the 3′terminus, one or more nucleotides that comprise a phosphorothioate linkage; and (iii) a 3′-block that prevent polymerase extension on the 3′ terminus of the blocking oligonucleotide; wherein the one or more blocking primers bind to the template sequences of non-desired fragments, thereby blocking amplification of the non-desired fragments by PCR. In a further embodiment, the one or more of the blocking oligonucleotides are from 15 nt to 100 nt in length. In yet a further embodiment, if the polymerase has 5′ to 3′ exonuclease activity, then the one or more of the blocking oligonucleotides comprise at the 5′ terminus, 1 to 5 nucleotides that comprise a phosphorothioate linkage. In another embodiment, if the polymerase has 3′ to 5′ proofreading activity, then the one or more of the blocking oligonucleotides comprise at the 3′ terminus, 1 to 5 nucleotides that comprise a phosphorothioate linkage. In yet another embodiment, the one or more blocking oligonucleotides comprise (i), (ii), and (iii): (i) at the 5′ terminus, 2 to 5 nucleotides that comprise a phosphorothioate linkage; and/or (ii) at the 3′terminus, 2 to 5 nucleotides that comprise a phosphorothioate linkage; and (iii) a 3′-block that prevent polymerase extension on the 3′ terminus of the blocking oligonucleotide. In another embodiment, the 3′-block is selected from a C₃-spacer, 3′ inverted bases, 3′ phosphorylation, 3′ dideoxy bases or 3′ non-complementary overhanging bases. In yet another embodiment, the amplified libraries comprise template sequences from CDNA. In a further embodiment, the amplified libraries comprise template sequences from gDNA. In a particular embodiment, the adapter sequences are from Y-shaped adapters that have been ligated to each end of a template sequence. In another embodiment, the one or more blocking oligonucleotides bind to template sequences from rRNAs and/or globin. In yet another embodiment, the one or more blocking oligonucleotides comprise a pool of blocking oligonucleotides that bind to template sequences from 18S rRNA, 5.8S rRNA, and/or 28S RNA. In a further embodiment, the one or more of the blocking oligonucleotides bind to template sequences from mtDNA. In yet a further embodiment, the amplified DNA or cDNA libraries are analyzed by using next generation sequencing. In a particular embodiment, the PCR amplification step is preceded by the following steps: obtaining an RNA sample; fragmenting the RNA; reverse transcribing the RNA fragments to cDNA; blunt ending the cDNA and adding an A nucleotide to the 3′ end of the blunt ended cDNA; and ligating the A-tailed cDNA with adapters comprising a non-complemented T nucleotide at the 3′ end. In a further embodiment, prior to reverse transcribing the RNA fragments to cDNA, the RNA sample is treated to deplete rRNA sequences from the RNA sample.

In a certain embodiment, the disclosure further provides a method to selectively deplete non-desirable fragments from amplified DNA or cDNA libraries by using one or more blocking oligonucleotides, comprising: amplifying in a polymerase chain reaction (PCR) reaction, a plurality of library fragments comprising a double stranded template sequence including adapter sequences, wherein a portion of the fragments comprise non-desirable fragments that contain template sequences that are not to be analyzed; wherein the PCR reaction comprises a plurality of fragments, a polymerase, dNTPs, PCR primers, and a pool of blocking oligonucleotides, wherein a portion of the pool of the blocking oligonucleotides bind to each strand of a template sequence of a non-desired fragment; wherein the one or more blocking primers bind to the template sequences of non-desired fragments, thereby blocking amplification of the non-desired fragments by PCR. In a further embodiment, the pool of blocking oligonucleotides are from 15 nt to 100 nt in length. In yet a further embodiment, the pool of blocking oligonucleotides comprise blocking oligonucleotides which bind to the strands of the template in a nonoverlapping and adjacent manner. In another embodiment, the pool of blocking oligonucleotides comprise blocking oligonucleotides that are reverse-complement to other blocking oligonucleotides. In yet another embodiment, the pool of blocking oligonucleotides comprise (i) and/or (ii), and (iii): (i) at the 5′ terminus, one or more nucleotides that comprise a phosphorothioate linkage; and/or (ii) at the 3′terminus, one or more nucleotides that comprise a phosphorothioate linkage; and (iii) a 3′-block that prevent polymerase extension on the 3′ terminus of the blocking oligonucleotide. In a further embodiment, if the polymerase has 5′ to 3′ exonuclease activity, then the one or more of the blocking oligonucleotides comprise at the 5′ terminus, 1 to 5 nucleotides that comprise a phosphorothioate linkage. In yet a further embodiment, if the polymerase has 3′ to 5′ proofreading activity, then the one or more of the blocking oligonucleotides comprise at the 3′ terminus, 1 to 5 nucleotides that comprise a phosphorothioate linkage. In a certain embodiment, the one or more blocking oligonucleotides comprise (i), (ii), and (iii): (i) at the 5′ terminus, 2 to 5 nucleotides that comprise a phosphorothioate linkage; (ii) at the 3′terminus, 2 to 5 nucleotides that comprise a phosphorothioate linkage; and (iii) a 3′-block that prevent polymerase extension on the 3′ terminus of the blocking oligonucleotide. In a further embodiment, the 3′-block is selected from a C₃-spacer, 3′ inverted bases, 3′ phosphorylation, 3′ dideoxy bases or 3′ non-complementary overhanging bases. In another embodiment, the amplified libraries comprise template sequences from CDNA. In yet another embodiment, the amplified libraries comprise template sequences from gDNA. In a further embodiment, the adapter sequences are from Y-shaped adapters that have been ligated to each end of a template sequence. In yet a further embodiment, the pool of blocking oligonucleotides bind to template sequences from rRNAs and/or globin. In another embodiment, the pool of blocking oligonucleotides bind to template sequences from 18S rRNA, 5.8S rRNA, and/or 28S RNA. In a further embodiment, the pool of blocking of blocking oligonucleotides bind to template sequences from mtDNA. In yet a further embodiment, the amplified DNA or cDNA libraries are analyzed by using next generation sequencing. In another embodiment, the PCR amplification step is preceded by the following steps: obtaining an RNA sample; fragmenting the RNA; reverse transcribing the RNA fragments to cDNA; blunt ending the cDNA and adding an A nucleotide to the 3′ end of the blunt ended cDNA; and ligating the A-tailed cDNA with adapters comprising a non-complemented T nucleotide at the 3′ end. In yet another embodiment, prior to reverse transcribing the RNA fragments to cDNA, the RNA sample is treated to deplete rRNA sequences from the RNA sample.

In a particular embodiment, the disclosure further provides a RNA-Seq based library preparation kit comprising one or more blocking oligonucleotides, wherein the one or more blocking oligonucleotides comprise (i) and/or (ii), and (iii): (i) at the 5′ terminus, one or more nucleotides that comprise a phosphorothioate linkage; and/or (ii) at the 3′terminus, one or more nucleotides that comprise a phosphorothioate linkage; and (iii) a 3′-block that prevent polymerase extension on the 3′ terminus of the blocking oligonucleotide; wherein the one or more blocking oligonucleotides bind to template sequences of non-desired library fragments, thereby blocking amplification of the non-desired library fragments by PCR. In a further embodiment, the library preparation kit further comprises: an A-tailing mix; an enhanced PCR mix; a ligation mix; a resuspension buffer; a stop ligation buffer; an Elute, Prime, Fragment High Concentration Mix; a First strand Synthesis Act D Mix; a reverse transcriptase; and a second strand master mix. In yet a further embodiment, the one or more of the blocking oligonucleotides are from 15 nt to 100 nt in length.

In a certain embodiment, the disclosure provides an RNA-Seq based library preparation kit comprising a pool of blocking oligonucleotides, wherein a portion of the pool of blocking oligonucleotides bind to each strand of a template sequence of a non-desired fragment in a nonoverlapping and adjacent manner, thereby blocking amplification of the non-desired library fragments by PCR. In a further embodiment, the library preparation kit further comprises: an A-tailing mix; an enhanced PCR mix; a ligation mix; a resuspension buffer; a stop ligation buffer; an Elute, Prime, Fragment High Concentration Mix; a First strand Synthesis Act D Mix; a reverse transcriptase; and a second strand master mix. In a further embodiment, the pool of the blocking oligonucleotides are from 15 nt to 100 nt in length. In yet a further embodiment, the pool of blocking oligonucleotides comprise (i) and/or (ii), and (iii): (i) at the 5′ terminus, one or more nucleotides that comprise a phosphorothioate linkage; and/or (ii) at the 3′terminus, one or more nucleotides that comprise a phosphorothioate linkage; and (iii) a 3′-block that prevent polymerase extension on the 3′ terminus of the blocking oligonucleotide. In a further embodiment, the 3′-block is selected from a C₃-spacer, 3′ inverted bases, 3′ phosphorylation, 3′ dideoxy bases or 3′ non-complementary overhanging bases.

The details of one or more embodiments of the disclosure are set forth in the accompanying drawings and the description below. Other features, objects, and advantages will be apparent from the description and drawings, and from the claims.

### BRIEF DESCRIPTION OF DRAWINGS

FIG. 1 presents workflow overviews for the traditional Total RNA workflow compared to the use of PCR clamps to deplete RNA-Seq libraries of rRNA fragments.

FIG. 2A-D provides an illustration of how the PCR clamps can be used to deplete sequencing libraries of unwanted fragments. (A) Key reagents in reaction: sequencing library composed of desired and non-desired fragments, PCR clamps, and PCR amplification primers. For simplicity, only 2 library fragment types are shown: one non-desired fragment targeted by the PCR clamps (red) and one fragment that is not targeted by the PCR clamps. Dark grey ends at library fragments represent universal adapter sequences. (B) Hybridization of PCR clamps and PCR primers: following denaturation by high temperature in PCR, reactions are cooled to allow annealing of PCR primers. Simultaneously, non-desired library fragments are targeted for removal by hybridizing with PCR clamps, while desired library fragments remain unbound by any PCR clamps. A key feature is that complete end-to-end hybridization of the PCR clamp to its target is not required. Thus, many non-desired library fragments can be targeted for depletion without a priori knowledge of their specific nature within a library. (C) Extension: thermostable polymerases extend from PCR primers to generate a copy of library fragments. PCR clamps bound to non-desired fragments cannot be completely copied due to blocking by bound PCR clamps. Desired library fragments are copied unimpeded by PCR clamps. (D) Final library: the final library is generated from exponential amplification of desired library fragments (grey), while non-desired library fragments (red) were inefficiently amplified. The result is a library that is “depleted” of non-desired library fragments.

FIG. 3 provides an overview of the exemplary PCR clamps that were designed to block amplification of rRNA genes. Design 1 provides for antiparallel and adjacent PCR clamps. Design 1+2 provides non-overlapping PCR clamps that incorporate Design 1 features with additional reverse-complement PCR clamps added in. Design 3 provides for overlapping antiparallel PCR clamps.

FIG. 4 shows that PCR clamps, as designed in Design 1 or Design 1_2, significantly reduced rRNA amplification transcripts when non-depleted total RNA was used. rRNA was decreased from ˜85% to 30% using PCR clamps in comparison to control (no PCR clamps).

FIG. 5 shows that PCR clamps, as designed in Design 1 or Design 1_2, further reduced rRNA in RPO enriched samples and in non-depleted, total RNA samples. DesignOffSet (Design 3) did not meaningfully affect rRNA enrichment in the RPO samples. Using Design 1 or Design 1_2 PCR clamps decreased rRNA enrichment from ˜20% to 18.

FIG. 6 demonstrates that PCR clamps, as designed in Design 1 or Design 1_2, reduced targeted rRNA in mRNA selected samples. Design 1 and 2 were able to further reduce BrRNA in mRNA selected samples from ˜1.5% rRNA to ˜0.25% rRNA

FIG. 7 provides Fragments Per Kilobase of transcript per Million mapped reads (FPKM) comparison between PCR clamps and RiboZero methods.

FIG. 8 demonstrates that samples using PCR clamps have high level expression correlation with FPKM R2 values>0.95 across different depletion methods.

FIG. 9 shows a trace of data generated from a probe panel with no optimization. Additional gains may be possible by optimizing probe design and workflow biochemistry.

FIG. 10 provides an exemplary embodiment of a PCR clamp (blocking Oligo) of the disclosure.

FIG. 11 provides examples of PCR clamps that can be generated from the sequences of 28S rRNA, 18S rRNA, 5.85rRNA, Mt12S rRNA and mt16S with PCR clamps designed to have a melting temperature of 75° C. or 80° C. Circles indicate gaps of sequence where there 80° C. PCR clamps cannot be generated from the rRNA sequence (as indicated in the Table).

FIG. 12 shows data from an rRNA-containing RNAseq data. The majority of the reads were blocked with PCR clamps with an 80° C. melting temperature.

FIG. 13 presents an overview of the PCR clamp study. (Top Panel) Overview of the 42 kbp human ribosomal DNA complete repeating unit (GenBank U13359.1). The three loci encoding highly abundant ribosomal RNAs (18S, 5.8S, and 28S) are noted in red. Additional features are shown in dark grey. (Bottom Panel) Closeup of the region containing the loci encoding the 18S, 5.8S and 28S rRNAs. The rRNA genes are noted in red. Two designs of PCR clamps are shown: Design 1 with alternating 80-mer PCR clamps tiled end-to-end. Every other PCR clamp is in an alternating 5′≥3′ orientation relative to the targeted rRNA gene (either lighter gray or darker gray). Design 2 contains PCR clamps in the same relative positions as Design 1, though each clamp is the reverse-complement sequence of Design 1.

The accompanying drawings, which are incorporated into and constitute a part of this specification, illustrate one or more embodiments of the disclosure and, together with the detailed description, serve to explain the principles and implementations of the disclosure.

### DETAILED DESCRIPTION

As used herein and in the appended claims, the singular forms “a,” “an,” and “the” include plural referents unless the context clearly dictates otherwise. Thus, for example, reference to “an oligonucleotide” includes a plurality of such oligonucleotides and reference to “the target sequence” includes reference to one or more target sequences, and so forth.

Also, the use of “or” means “and/or” unless stated otherwise. Similarly, “comprise,” “comprises,” “comprising,” “include,” “includes,” “including,” “have,” “haves,” and “having” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The expression “amplification” or “amplifying” refers to a process by which extra or multiple copies of a particular polynucleotide are formed. Amplification includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., “PCR protocols: a guide to method and applications” Academic Press, Incorporated (1990) (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting amplification reaction are commercially available. Primers useful to amplify sequences from a particular gene region are preferably complementary to, and hybridize specifically to sequences in the target region or in its flanking regions and can be prepared using the polynucleotide sequences provided herein. Nucleic acid sequences generated by amplification can be sequenced directly.

A “blocking oligonucleotide” as used herein refers to a nucleic acid molecule that can specifically bind to at least one of the one or more undesirable nucleic acid species, whereby the binding between the blocking oligonucleotide and the one or more undesirable nucleic acid species can reduce or prevent the amplification or extension (e.g., reverse transcription) of the one or more undesirable nucleic acid species. For example, the blocking oligonucleotide can comprise a nucleic acid sequence capable of hybridizing with one or more undesirable nucleic acid species. In some embodiments, a plurality of blocking oligonucleotides can be provided. The plurality of blocking oligonucleotides can specifically bind to at least 1, at least 2, at least 5, at least 10, at least 100, at least 1,000 or more of the one or more undesirable nucleic acid species. Further, a plurality of different blocking oligonucleotides can specifically bind to at least 1, at least 2, at least 5, at least 10, at least 20, at least 100 different sites on the same undesirable nucleic acid species in parallel, antiparallel, spaced or sequential sites on the undesirable nucleic acid species. The location at which a blocking oligonucleotide specifically binds to an undesirable nucleic acid species can vary. For example, a blocking oligonucleotide can specifically bind to a sequence close to the 5′ end of the undesirable nucleic acid species. In some embodiments, the blocking oligonucleotide can specifically bind to within 10 nt, 20 nt, 30 nt, 40 nt, 50 nt, 100 nt, 200 nt, 300 nt, 400 nt, 500 nt, or 1,000 nt of the 5′ end of at least one of the one or more undesirable nucleic acid species. In some embodiments, a blocking oligonucleotide can specifically bind to a sequence close to the 3′ end of the undesirable nucleic acid species. For example, the blocking oligonucleotide can specifically bind to within 10 nt, 20 nt, 30 nt, 40 nt, 50 nt, 100 nt, 200 nt, 300 nt, 400 nt, 500 nt, 1,000 nt of the 3′ end of at least one of the one or more undesirable nucleic acid species. As another example, blocking oligonucleotide can specifically binds to a sequence in the middle portion of the undesirable nucleic acid species. In some embodiments, the blocking oligonucleotide can specifically bind to within 10 nt, 20 nt, 30 nt, 40 nt, 50 nt, 100 nt, 200 nt, 300 nt, 400 nt, 500 nt, 1,000 nt from the middle point of at least one of the one or more undesirable nucleic acid species. In some embodiments, blocking oligonucleotides can bind at multiple positions between the 5′ and the 3′ end of the undesirable nucleic acid species.

In some embodiments, the binding between the blocking oligonucleotide (s) and the undesirable nucleic acid species can reduce amplification and/or extension of the undesirable nucleic acid species by at least 108, at least 208, at least 30%, at least 40%, at least 508, at least 60%, at least 708, at least 80%, at least 90%, at least 95%, at least 98%, at least 998, or 100%.

It is contemplated that the blocking oligonucleotide may reduce the amplification and/or extension of the undesirable nucleic acid species by, for example, forming a hybridization complex with the undesirable nucleic acid species such that the complex has a high melting temperature (Tₘ), thus not allowing the blocking oligonucleotide to function as a primer for a reverse transcriptase or a polymerase, or a combination thereof. In some embodiments, the blocking oligonucleotide (s) can have a Tₘ of 48° C., 49° ° C., 50° C., 51° ° C., 52° C., 53° C., 54° C., 55° ° C., 56° ° C., 57° ° C., 58° ° C., 59° C., 60° ° C., 61° C., 62° ° C., 63° C., 64° C., 65° ° C., 70° ° C., 75° ° C., 80° ° C., or a range (e.g., 50° C. to 60° C.) that includes or is between any two of the foregoing temperatures.

The blocking oligonucleotide can, in some embodiments, comprise one or more non-natural nucleotides. Non-natural nucleotides can be, for example, photolabile or triggerable nucleotides. Examples of non-natural nucleotides can include, but are not limited to, peptide nucleic acid (PNA), morpholino and locked nucleic acid (LNA), as well as glycol nucleic acid (GNA) and threose nucleic acid (TNA). In some embodiments, the blocking oligonucleotide is a chimeric oligonucleotide, such as an LNA/PNA/DNA chimera, an LNA/DNA chimera, a PNA/DNA chimera, a GNA/DNA chimera, a TNA/DNA chimera, or a combination thereof.

A blocking oligonucleotide can have a length that is, is about 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 35 nt, 40 nt, 45 nt, 50 nt, 60 nt, 70 nt, 80 nt, 90 nt, 100 nt, 200 nt, or a range (e.g., 17 nt to 30 nt) that includes or is between any two of foregoing nucleotide lengths.

The melting temperature (Tₘ) of a blocking oligonucleotide can be modified, in some embodiments, by adjusting the length of the blocking oligonucleotide. In some embodiments, the Tₘ of a blocking oligonucleotide is modified by the number of DNA residues in the blocking oligonucleotide that comprises an LNA/DNA chimera or a PNA/DNA chimera. For example, a blocking oligonucleotide that comprises an LNA/DNA chimera or a PNA/DNA chimera can have a percentage of DNA residues that is about 10%, 15%, 20%, 25%, 30%, 35%, 40%, 45%, 50%, 60%, 70%, 80%, 90%, 95%, 99% or a range between any two of the above values.

In some embodiments, a blocking oligonucleotide can be designed to be incapable of functioning as a primer or probe for an amplification and/or extension reaction. For example, the blocking oligonucleotide may be incapable of function as a primer for a reverse transcriptase or a polymerase. For example, a blocking oligonucleotide that comprises an LNA/DNA chimera or a PNA/DNA chimera can be designed to have a certain percentage of LNA or PNA residues, or to have LNA or PNA residues on certain locations, such as close to or at the 3′ end, 5′ end, or in the middle portion of the oligonucleotide. In some embodiments, a blocking oligonucleotide that comprises an LNA/DNA chimera or a PNA/DNA chimera can have a percentage of LNA or PNA residues that is about 10%, 15%, 20%, 25%, 30%, 35%, 40%, 45%, 50%, 60%, 70%, 80%, 90%, or a range between any two of the above values.

The term “cDNA library” refers to a collection of cloned complementary DNA (cDNA) fragments, which together constitute some portion of the transcriptome of a single cell or a plurality of single cells. cDNA is produced from fully transcribed mRNA found in a cell and therefore contains only the expressed genes of a single cell or when pooled together the expressed genes from a plurality of single cells.

As used herein, the term “complementary” can refer to the capacity for precise pairing between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind (e.g., there are one or more mismatches between a blocking oligo and a complementary target), or it may be complete when total complementarity exists between the single-stranded molecules (e.g., there are no mismatches between a blocking oligo and a complementary target). A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence. As used herein, the terms “complement”, “complementary”, and “reverse complement” can be used interchangeably. It is understood from the disclosure that if a molecule can hybridize to another molecule, it may be the complement of the molecule that is hybridizing.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell.

The term “homologs” used with respect to an original enzyme or gene of a first family or species refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

As used herein, two polynucleotides, oligonucleotides, peptides, polypeptides or proteins (or a fragment of any of the foregoing) are substantially homologous when the nucleic acid or amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When hybridization occurs in an antiparallel configuration between two single-stranded polynucleotides, the reaction is called “annealing” and those polynucleotides are described as “complementary”. A double-stranded polynucleotide can be complementary or homologous to another polynucleotide, if hybridization can occur between one of the strands of the first polynucleotide and the second. Complementarity or homology (the degree that one polynucleotide is complementary with another) is quantifiable in terms of the proportion of bases in opposing strands that are expected to form hydrogen bonding with each other, according to generally accepted base-pairing rules.

The terms “oligonucleotide” and “polynucleotide” are used interchangeably and refer to a polymeric form of nucleotides of any length, either deoxyribonucleotides or ribonucleotides or analogs thereof. Polynucleotides can have any three-dimensional structure and can perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide (e.g., a blocking oligonucleotide) can comprise modified nucleotides, such as methylated nucleotides and nucleotide analogs. The term also refers to both double- and single-stranded molecules. Unless otherwise specified or required, any embodiment of this disclosure that comprises a polynucleotide encompasses both the double-stranded form and each of two complementary single-stranded forms known or predicted to make up the double-stranded form.

A nucleic acid useful in the methods and compositions disclosed herein can contain a non-natural sugar moiety in the backbone. Exemplary sugar modifications include but are not limited to 2′ modifications such as addition of halogen, alkyl, substituted alkyl, —SH, —SCH₃, —OCN, —Cl, —Br, —CN, —CF₃, —OCF₃, —SO₂CH₃, —OSO₂, —SO₃, —CH₃, —ONO₂, —NO₂, —N₃, —NH₂, substituted silyl, and the like. Similar modifications can also be made at other positions on the sugar, particularly the 3′ position of the sugar on the 3′ terminal nucleotide or in 2′-5′ linked oligonucleotides and the 5′ position of 5′ terminal nucleotide. Nucleic acids, nucleoside analogs or nucleotide analogs having sugar modifications can be further modified to include a reversible blocking group, peptide linked label or both. In those embodiments where the above-described 2′ modifications are present, the base can have a peptide linked label.

A nucleic acid useful in the methods and compositions disclosed herein also can include native or non-native bases. In this regard a native deoxyribonucleic acid can have one or more bases selected from the group consisting of adenine, thymine, cytosine or guanine and a ribonucleic acid can have one or more bases selected from the group consisting of uracil, adenine, cytosine or guanine. Exemplary non-native bases that can be included in a nucleic acid, whether having a native backbone or analog structure, include, without limitation, inosine, xathanine, hypoxathanine, isocytosine, isoguanine, 5-methylcytosine, 5-hydroxymethyl cytosine, 2-aminoadenine, 6-methyl adenine, 6-methyl guanine, 2-propyl guanine, 2-propyl adenine, 2-thioLiracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine. 3-deazaguanine, 3-deazaadenine or the like. A particular embodiment can utilize isocytosine and isoguanine in a nucleic acid in order to reduce non-specific hybridization, as generally described in U.S. Pat. No. 5,681,702.

A non-native base used in a nucleic acid of the disclosure can have universal base pairing activity, wherein it is capable of base pairing with any other naturally occurring base. Exemplary bases having universal base pairing activity include 3-nitropyrrole and 5-nitroindole. Other bases that can be used include those that have base pairing activity with a subset of the naturally occurring bases such as inosine, which base pairs with cytosine, adenine or uracil.

A polynucleotide is composed of a specific sequence of four nucleotide bases: adenine (A); cytosine (C); guanine (G); thymine (T); and uracil (U) for thymine when the polynucleotide is RNA. Thus, the term polynucleotide sequence is the alphabetical representation of a polynucleotide molecule. This alphabetical representation can be input into databases in a computer having a central processing unit and used for bioinformatics applications such as functional genomics and homology searching.

The term “library” refers to a collection or plurality of template molecules, which at their 5′ and 3′ ends typically comprise added adapter sequences. Use of the term “library” to refer to a collection or plurality of template molecules should not be taken to imply that the templates making up the library are derived from a particular source, or that the “library” has a particular composition. By way of example, use of the term “library” should not be taken to imply that the individual templates within the library must be of different nucleotide sequence or that the templates be related in terms of sequence and/or source.

As used herein, the term “locked nucleic acid” or “LNA” refers to a modified RNA nucleotide. The ribose moiety of an LNA nucleotide is modified with an extra bridge connecting the 2′ oxygen and 4′ carbon. The bridge “locks” the ribose in the 3′-endo (North) conformation. Some of the advantages of using LNAs in the methods of the disclosure include increasing the thermal stability of duplexes, increased target specificity and resistance from exo- and endonucleases.

In various embodiments the disclosure encompasses formation of so-called “monotemplate” libraries, which comprise multiple copies of a single type of template molecule, each having added adapter sequences at their 5′ ends and their 3′ ends, as well as “complex” libraries wherein many, if not all, of the individual template molecules comprise different target sequences (as defined below), where each template molecule has added on adapter sequences at their 5′ ends and their 3′ ends. Such complex template libraries may be prepared using the method of the disclosure starting from a complex mixture of target polynucleotides such as (but not limited to) random genomic DNA fragments, cDNA etc. The disclosure also extends to “complex” libraries formed by mixing together several individual “monotemplate” libraries, each of which has been prepared separately using the method of the disclosure starting from a single type of target molecule (i.e., a monotemplate). In a particular embodiment more than 50%, or more than 608, or more than 70%, or more than 80%, or more than 908, or more than 95% of the individual polynucleotide templates in a complex library may comprise different target sequences.

As used herein, a “plurality” refers to a population of molecules and can include any number of molecules desired to be analyzed.

As used herein, a “peptide nucleic acid” or “PNA” refers to an artificially synthesized polymer similar to DNA or RNA, wherein the backbone is composed of repeating N-(2-aminoethyl)-glycine units linked by peptide bonds. The backbone of a PNA is substantially non-ionic under neutral conditions, in contrast to the highly charged phosphodiester backbone of naturally occurring nucleic acids. This provides two non-limiting advantages. First, the PNA backbone exhibits improved hybridization kinetics. Secondly, PNAs have larger changes in the melting temperature (Tm) for mismatched versus perfectly matched base pairs. DNA and RNA typically exhibit a 2-4° C. drop in Tₘ for an internal mismatch. With the non-ionic PNA backbone, the drop is closer to 7-9° C. This can provide for better sequence discrimination. Similarly, due to their non-ionic nature, hybridization of the bases attached to these backbones is relatively insensitive to salt concentration.

A “primer” a short polynucleotide, generally with a free 3′-OH group that binds to a target or template potentially present in a sample of interest by hybridizing with the target, and thereafter promoting polymerization of a poly nucleotide complementary to the target. Primers of the disclosure are comprised of nucleotides ranging from 17 to 30 nucleotides. In one embodiment, the primer is at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively at least 50 nucleotides, or alternatively at least 75 nucleotides or alternatively at least 100 nucleotides.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. Furthermore, in general, cells from any population can be used in the methods, such as a population of prokaryotic or eukaryotic single celled organisms including bacteria or yeast. In some embodiments, the method of preparing the cDNA library can include the step of obtaining single cells. A single cell suspension can be obtained using standard methods known in the art including, for example, enzymatically using trypsin or papain to digest proteins connecting cells in tissue samples or releasing adherent cells in culture, or mechanically separating cells in a sample. Single cells can be placed in any suitable reaction vessel in which single cells can be treated individually. For example, a 96-well plate, such that each single cell is placed in a single well.

Methods for manipulating single cells are known in the art and include fluorescence activated cell sorting (FACS), micromanipulation and the use of semi-automated cell pickers (e.g., the Quixell™ cell transfer system from Stoelting Co.). Individual cells can, for example, be individually selected based on features detectable by microscopic observation, such as location, morphology, or reporter gene expression.

Use of the term “template” to refer to individual polynucleotide molecules in the library merely indicates that one or both strands of the polynucleotides in the library are capable of acting as templates for template-dependent nucleic-acid polymerization catalyzed by a polymerase. Use of this term should not be taken as limiting the scope of the disclosure to libraries of polynucleotides which are actually used as templates in a subsequent enzyme-catalyzed polymerization reaction.

The term “unmatched region” refers to a region of the adapter wherein the sequences of the two polynucleotide strands forming the adapter exhibit a degree of non-complementarity such that the two strands are not capable of annealing to each other under standard annealing conditions for a PCR reaction. The two strands in the unmatched region may exhibit some degree of annealing under standard reaction conditions for an enzyme-catalyzed ligation reaction, provided that the two strands revert to single stranded form under annealing conditions.

The pooled cDNA samples can be amplified by polymerase chain reaction (PCR) including emulsion PCR and single primer PCR in the methods described herein. For example, the cDNA samples can be amplified by single primer PCR. The CDNA synthesis primer can comprise a 5′ amplification primer sequence (APS), which subsequently allows the first strand of cDNA to be amplified by PCR using a primer that is complementary to the 5′ APS. The template switch oligonucleotide can also comprise a 5′ APS, which can be at least 70% identical, at least 80% identical, at least 90% identical, at least 95% identical, or 70%, 80%, 90% or 100% identical to the 5′ APS in the cDNA synthesis primer. This means that the pooled cDNA samples can be amplified by PCR using a single primer (i.e., by single primer PCR), which exploits the PCR suppression effect to reduce the amplification of short contaminating amplicons and primer-dimers (Dai et al., J Biotechnol 128(3): 435-43 (2007)). As the two ends of each amplicon are complementary, short amplicons will form stable hairpins, which are poor templates for PCR. This reduces the amount of truncated cDNA and improves the yield of longer cDNA molecules. The 5′ APS can be designed to facilitate downstream processing of the cDNA library. For example, if the cDNA library is to be analyzed by a particular sequencing method, e.g., Life Technology's SOLID sequencing technology, or Illumina's Genome Analyzer, the 5′ APS can be designed to be identical to the primers used in these sequencing methods. For example, the 5′ APS can be identical to the SOLID P1 primer, and/or a SOLID P2 sequence inserted in the cDNA synthesis primer, so that the P1 and P2 sequences required for SOLID sequencing are integral to the amplified library.

Another exemplary method for amplifying pooled cDNA includes PCR. PCR is a reaction in which replicate copies are made of a target polynucleotide using a pair of primers or a set of primers consisting of an upstream and a downstream primer, and a catalyst of polymerization, such as a DNA polymerase, and typically a thermally-stable polymerase enzyme. Methods for PCR are well known in the art, and taught, for example in MacPherson et al. (1991) PCR 1: A Practical Approach (IRL Press at Oxford University Press). All processes of producing replicate copies of a polynucleotide, such as PCR or gene cloning, are collectively referred to herein as replication. A primer can also be used as a probe in hybridization reactions, such as Southern or Northern blot analyses.

For emulsion PCR, an emulsion PCR reaction is created by vigorously shaking or stirring a “water in oil” mix to generate millions of micron-sized aqueous compartments. The DNA library is mixed in a limiting dilution either with the beads prior to emulsification or directly into the emulsion mix. The combination of compartment size and limiting dilution of beads and target molecules is used to generate compartments containing, on average, just one DNA molecule and bead (at the optimal dilution many compartments will have beads without any target) To facilitate amplification efficiency, both an upstream (low concentration, matches primer sequence on bead) and downstream PCR primers (high concentration) are included in the reaction mix. Depending on the size of the aqueous compartments generated during the emulsification step, up to 3×10⁹ individual PCR reactions per μl can be conducted simultaneously in the same tube. Essentially each little compartment in the emulsion forms a micro-PCR reactor. The average size of a compartment in an emulsion range from sub-micron in diameter to over 100 microns, depending on the emulsification conditions.

“Identity,” “homology” or “similarity” are used interchangeably and refer to the sequence similarity between two nucleic acid molecules. Identity can be determined by comparing a position in each sequence which can be aligned for purposes of comparison. When a position in the compared sequence is occupied by the same base or amino acid, then the molecules are homologous at that position. A degree of identity between sequences is a function of the number of matching or identical positions shared by the sequences. An unrelated or non-homologous sequence shares less than 40% identity, or alternatively less than 25% identity, with one of the sequences disclosed herein.

A polynucleotide has a certain percentage (for example, 60%, 65%, 70%, 75%, 80%, 85%, 90%, 95%, 98% or 99%) of “sequence identity” to another sequence means that, when aligned, that percentage of bases are the same in comparing the two sequences. This alignment and the percent sequence identity or homology can be determined using software programs known in the art, for example those described in Ausubel et al., Current Protocols in Molecular Biology, John Wiley & Sons, New York, N.Y., (1993). Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein SPupdate+PIR. Details of these programs can be found at the National Center for Biotechnology Information.

Sequence homology for polypeptides, which can also be referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A typical algorithm used to compare a molecular sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, 1990, hereby incorporated herein by reference). For example, percent sequence identity between amino acid sequences can be determined using FASTA with its default parameters (a word size of 2 and the PAM250 scoring matrix), as provided in GCG Version 6.1, hereby incorporated herein by reference.

The method of preparing a cDNA library described herein can further comprise processing the cDNA library to obtain a library suitable for sequencing. As used herein, a library is suitable for sequencing when the complexity, size, purity or the like of a cDNA library is suitable for the desired screening method. In particular, the cDNA library can be processed to make the sample suitable for any high-throughput screening methods, such as Life Technology's SOLID sequencing technology, Oxford's Nanopore DNA sequencing technology, or Illumina's cluster generation and sequencing technologies. As such, the cDNA library can be processed by fragmenting the cDNA library (e.g., with DNase) to obtain a short-fragment 5′-end library. Adapters can be added to the cDNA, e.g., at one or both ends to facilitate sequencing of the library. The cDNA library can be further amplified, e.g., by PCR, to obtain a sufficient quantity of cDNA for sequencing.

Embodiments of the disclosure provide a cDNA library produced by any of the methods described herein. This cDNA library can be sequenced to provide an analysis of gene expression in single cells or in a plurality of single cells.

Embodiments of the disclosure also provide a method for analyzing gene expression in a plurality of single cells, the method comprising the steps of preparing a cDNA library using the method described herein and sequencing the cDNA library. A “gene” refers to a poly nucleotide containing at least one open reading frame (ORF) that is capable of encoding a particular polypeptide or protein after being transcribed and translated. Any of the polynucleotide sequences described herein can be used to identify larger fragments or full-length coding sequences of the gene with which they are associated. Methods of isolating larger fragment sequences are known to those of skill in the art.

The cDNA library can be sequenced by any suitable screening method. In particular, the cDNA library can be sequenced using a high-throughput screening method, such as Life Technology's SOLID sequencing technology, Oxford's Nanopore DNA sequencing technology, or Illumina's cluster generation and sequencing technologies. In one embodiment, the cDNA library can be shotgun sequenced. The number of reads can be at least 10,000, at least 1 million, at least 10 million, at least 100 million, or at least 1000 million. In another embodiment, the number of reads can be from 10,000 to 100,000, or alternatively from 100,000 to 1 million, or alternatively from 1 million to 10 million, or alternatively from 10 million to 100 million, or alternatively from 100 million to 1000 million. A “read” is a length of continuous nucleic acid sequence obtained by a sequencing reaction.

Next-generation sequencing (NGS) libraries often contain abundant sequences with little biological significance, such as ribosomal RNA sequences in transcriptomic libraries, host sequences in microbiome or metagenomic libraries, or majority allele sequences in somatic variant detection applications. In RNA-seq libraries, for example, ribosomal RNA (rRNA) sequences can make up 95% or more of total reads; for most applications, these reads are uninformative and are discarded during secondary analysis. The flow cell ‘real estate’ taken up by these sequences can add significantly to the cost of sequencing, particularly for count-based applications or detection of rare fragments where greater sequencing depth is required to sufficiently sample the species of interest.

In all organisms, ribosomal RNAs (rRNAs), structural components of highly abundant ribosomes compose the vast majority of all RNA. Without selectively depleting the RNA sample of these ribosomal RNAs, the resulting NGS library is composed largely of fragments representing rRNA, which is of little use or scientific interest to the end user. Thus, rRNAs must be depleted from the sample prior to library construction. Current methods for depletion of abundant sequences, such as hybridization pull-down of rRNA (e.g., RiboZero, RiboMinus) or enzymatic digestion (e.g., RNaseH, CRISPR) perform well for high-quality, high-input samples, but often show poor performance with lower-quality, less abundant inputs encountered in clinically-relevant sample types such as formalin fixed/paraffin-embedded (FFPE) tissue and plasma-derived circulating RNA (C-RNA). Alternatively, sequence-specific enrichment approaches (e.g., exome capture) show better performance for low-input samples, but are restricted by the need to pre-specify a set of targets. This limits their utility for detecting rare transcript isoforms and non-coding RNAs that may be useful biomarkers. Additionally, these treatments to remove rRNA work directly on the sample, composed of chemically labile RNA, and introduce the risk of sample damage. Furthermore, these methods to reduce rRNA are only applicable to the RNA sample itself, and once the sample has been converted into library the same methods for rRNA capture or depletion are not applicable.

The use of one or more blocking oligonucleotides to reduce the abundance of non-desirable library fragments is described herein. The methods of the disclosure are extremely facile for the end user, requiring no additional library preparation steps and the addition of one or more oligonucleotides. The methods described herein act on created libraries, rather than on the sample directly, reducing the risk of damage to the original polynucleotide sample.

As shown in the studies presented herein, the methods of the disclosure significantly reduced rRNA for RNA-Seq technologies. Similar results would be expected when the methods of the disclosure apply to other library preparation (e.g., ds DNA libraries) where non-desirable library fragments are generated. Examples of other potential uses include, but are not limited to, the removal of globin RNAs, mitochondrial DNA fragments, housekeeping gene fragments from libraries, nonhost genetic material, and other scenarios where depletion of host or other abundant nucleic acids are desirable for production of more focused and data-rich NGS libraries.

Accordingly, the methods, compositions and kits of the disclosure can be used with DNA libraries generated from gDNA or other DNA sources. In such a case, the library generation would utilize standard methodologies, except for the PCR amplification step to make a DNA sequencing library from adapter/template constructs. In particular, one or more blocking oligonucleotides of the disclosure would be added as a component to the PCR amplification step to make a DNA sequencing library.

Various non-limiting specific embodiments of the method disclosed herein will now be described in further detail with reference to the accompanying drawings. Features described as being preferred in relation to one specific embodiment apply mutatis mutandis to other specific embodiments of the disclosure unless stated otherwise.

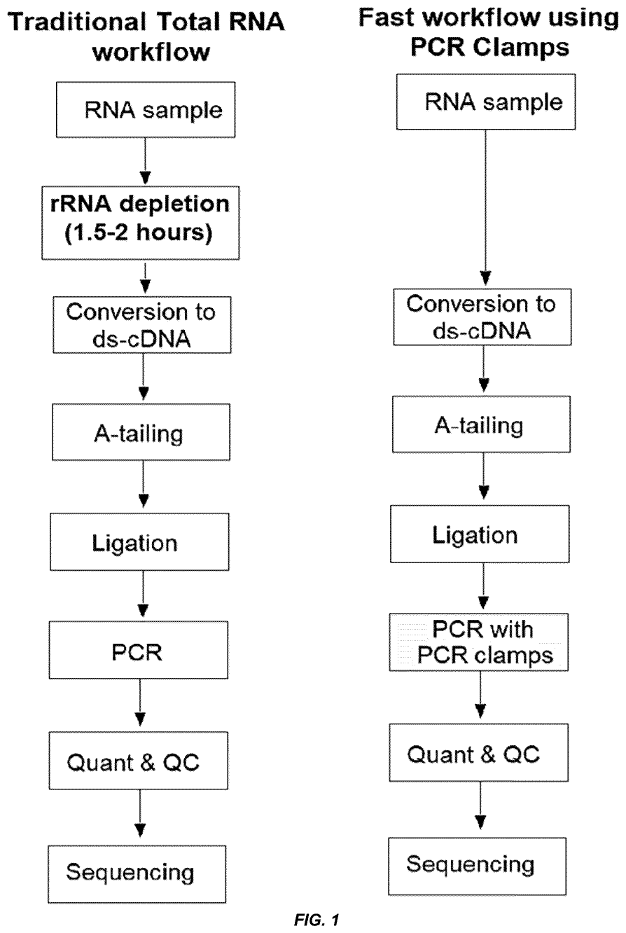

FIG. 1 illustrates the process traditionally used to generate a template library for sequencing from total RNA. The library preparation from total RNA is common to all major sequencing platforms, including those from Illumina™, Life Technologies™, and Oxford Nanopore™.

As shown in FIG. 1, total RNA sample is isolated from a sample using methodologies like those described herein. The total RNA is typically treated to remove rRNA by performing an rRNA depletion step. Current methods for depletion of rRNA, include hybridization pull-down of rRNA (e.g., RiboZero™, RiboMinus™) or enzymatic digestion (e.g., RNaseH, CRISPR). The above rRNA depletion methods can be lengthy (1.5-2 hours) and involve multiple subcomponents and steps. These depletion methods perform well for high-quality, high-input samples, but often show poor performance with lower-quality, less abundant inputs encountered in clinically-relevant sample types such as formalin-fixed/paraffin-embedded (FFPE) tissue and plasma-derived circulating RNA (C-RNA). Alternatively, sequence-specific enrichment approaches (e.g., exome capture) show better performance for low-input samples, but are restricted by the need to pre-specify a set of targets. This limits their utility for detecting rare transcript isoforms and non-coding RNAs that may be useful biomarkers. Further, the depletion methods for removing rRNA and other non-desired RNAs must be performed on the RNA sample itself. RNA is a labile nucleic acid and sensitive to handling, storage conditions, and RNase activity. It should be noted, that incomplete depletion of rRNA and other non-desired RNA using the above methods cannot be remedied in subsequent steps once it is converted into the library.

In direct contrast, the disclosure provides for a new, and innovative method to deplete non-desired nucleotide sequences using one or more blocking oligonucleotides (i.e., PCR clamps). Considerations for designing the blocking oligonucleotides are further described herein.

FIG. 1 illustrates an RNA-Seq process standardly used to generate a template library for sequencing from RNA. FIG. 1 further illustrates an RNA-Seq process that has been modified to incorporate one or more blocking oligonucleotides of the disclosure. RNA-Seq (named as an abbreviation of “RNA sequencing”) is a sequencing technique which uses next-generation sequencing (NGS) to reveal the presence and quantity of RNA in a biological sample at a given moment, analyzing the continuously changing cellular transcriptome.

Specifically, RNA-Seq facilitates the ability to look at alternative gene spliced transcripts, post-transcriptional modifications, gene fusion, mutations/SNPs and changes in gene expression over time, or differences in gene expression in different groups or treatments. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries. Recent advances in RNA-Seq include single cell sequencing and in situ sequencing of fixed tissue.

Prior to RNA-Seq, gene expression studies were done with hybridization-based microarrays. Issues with microarrays include cross-hybridization artifacts, poor quantification of lowly and highly expressed genes, and needing to know the sequence a priori. Because of these technical issues, transcriptomics transitioned to sequencing-based methods. These progressed from Sanger sequencing of Expressed Sequence Tag libraries, to chemical tag-based methods (e.g., serial analysis of gene expression), and finally to the current technology, next-gen sequencing of cDNA (notably RNA-Seq). Next generation sequencing (NGS) typically requires library preparation, where known adapter DNA sequences are added to the target nucleotides to be sequenced. Traditionally, this requires that RNA is converted to cDNA, fragmented, end-repaired, and then ligated to the adapter DNA (e.g., see FIG. 1). This library preparation is common to all major sequencing platforms, including those from Illumina™, Pacific Biosciences™, and Oxford Nanopore™.

As shown in FIG. 1, RNA is isolated from a sample. In a particular embodiment, RNA can be isolated from cells by lysing the cells. Lysis can be achieved by, for example, heating the cells, or by the use of detergents or other chemical methods, or by a combination of these. However, any suitable lysis method known in the art can be used. A mild lysis procedure can advantageously be used to prevent the release of nuclear chromatin, thereby avoiding genomic contamination of the cDNA library, and to minimize degradation of mRNA. For example, heating the cells at 72° C. for 2 minutes in the presence of Tween-20 is sufficient to lyse the cells while resulting in no detectable genomic contamination from nuclear chromatin. Alternatively, cells can be heated to 65° C. for 10 minutes in water (Esumi et al., Neurosci Res 60 (4): 439-51 (2008)); or 70° C. for 90 seconds in PCR buffer II (Life Technology) supplemented with 0.5% NP-40 (Kurimoto et al., Nucleic Acids Res 34 (5): e42 (2006)); or lysis can be achieved with a protease such as Proteinase K or by the use of chaotropic salts such as guanidine isothiocyanate (U.S. Publication No. 2007/0281313).

DNase is typically added to the RNA sample. DNase reduces the amount of genomic DNA. The amount of RNA degradation is checked with gel and capillary electrophoresis and is used to assign an RNA integrity number to the sample. This RNA quality and the total amount of starting RNA are taken into consideration during the subsequent library preparation, sequencing, and analysis steps. RNA can be isolated with good yield and of high quality using any number of commercially available kits such as kits from Qiagen or Ambion, Lucigen MasterPure Kits, etc. or using specific RNA isolation reagents, like TRIzol. The RNA integrity number should be greater than 8. RNA can be quantified using a fluorometric-based method, like Ribo-green.

As shown in FIG. 1, the RNA is then typically enriched by polyA selection or treated to deplete the RNA of rRNA samples. Current methods for depletion of abundant sequences, such as hybridization pull-down of rRNA (e.g., RiboZero, RiboMinus) or enzymatic digestion (e.g., RNaseH, CRISPR) perform well for high-quality, high-input samples, but often show poor performance with lower-quality, less abundant inputs encountered in clinically-relevant sample types such as formalin fixed/paraffin-embedded (FFPE) tissue and plasma-derived circulating RNA (C-RNA). Alternatively, sequence-specific enrichment approaches (e.g., exome capture) show better performance for low-input samples, but are restricted by the need to pre-specify a set of targets. This limits their utility for detecting rare transcript isoforms and non-coding RNAs that may be useful biomarkers. Typically, it takes 1 to 2 hours to deplete an RNA sample of rRNA.

After the RNA is treated to enrich the RNA sample with desired templates, the RNA is reverse transcribed into cDNA. Optionally, the RNA can be fragmented and size selected prior to conversion to cDNA. Fragmentation and size selection are performed to purify sequences that are the appropriate length for the sequencing machine. The RNA, CDNA, or both are fragmented with enzymes, sonication, or nebulizers. Fragmentation of the RNA reduces 5′ bias of randomly primed-reverse transcription and the influence of primer binding sites, with the downside that the 5′ and 3′ ends are converted to cDNA less efficiently. Fragmentation is followed by size selection, where either small sequences are removed or a tight range of sequence lengths are selected. Because small RNAs like miRNAs are lost, these are analyzed independently.

As shown in FIG. 1, treated RNA is converted into cDNA. CDNA is typically synthesized from mRNA by reverse transcription. Methods for synthesizing cDNA from small amounts of mRNA, including from single cells, have previously been described (Kurimoto et al., Nucleic Acids Res 34 (5): e42 (2006): Kurimoto et al., Nat Protoc 2 (3): 739-52 (2007); and Esumi et al., Neurosci Res 60 (4): 439-51 (2008)). In order to generate an amplifiable cDNA, these methods introduce a primer annealing sequence at both ends of each cDNA molecule in such a way that the cDNA library can be amplified using a single primer. The Kurimoto method uses a polymerase to add a 3′ poly-A tail to the cDNA strand, which can then be amplified using a universal oligo-T primer. In contrast, the Esumi method uses a template switching method to introduce an arbitrary sequence at the 3′ end of the cDNA, which is designed to be reverse complementary to the 3′ tail of the cDNA synthesis primer. Again, the cDNA library can be amplified by a single PCR primer. Single-primer PCR exploits the PCR suppression effect to reduce the amplification of short contaminating amplicons and primer-dimers (Dai et al., J Biotechnol 128 (3): 435-43 (2007)). As the two ends of each amplicon are complementary, short amplicons will form stable hairpins, which are poor templates for PCR. This reduces the amount of truncated cDNA and improves the yield of longer cDNA molecules.

In a particular embodiment, the synthesis of the first strand of the cDNA can be directed by a cDNA synthesis primer (CDS) that includes an RNA complementary sequence (RCS). In another embodiment, the RCS is at least partially complementary to one or more mRNA in an individual mRNA sample. This allows the primer, which is typically an oligonucleotide, to hybridize to at least some mRNA in an individual mRNA sample to direct cDNA synthesis using the mRNA as a template. The RCS can comprise oligo (dT), or be gene family-specific, such as a sequence of nucleic acids present in all or a majority related gene, or can be composed of a random sequence, such as random hexamers. To avoid the cDNA synthesis primer priming on itself and thus generating undesired side products, a non-self-complementary semi-random sequence can be used. For example, one letter of the genetic code can be excluded, or a more complex design can be used while restricting the cDNA synthesis primer to be non-self-complementary.

The RCS can also be at least partially complementary to a portion of the first strand of cDNA, such that it is able to direct the synthesis of a second strand of cDNA using the first strand of the cDNA as a template. Thus, following first strand synthesis, an RNase enzyme (e.g., an enzyme having RNaseH activity) can be added after synthesis of the first strand of DNA to degrade the RNA strand and to permit the cDNA synthesis primer to anneal again on the first strand to direct the synthesis of a second strand of cDNA. For example, the RCS could comprise random hexamers, or a non-self-complementary semi-random sequence (which minimizes self-annealing of the cDNA synthesis primer).

A template switch oligonucleotide (TSO) that includes a portion which is at least partially complementary to a portion of the 3′ end of the first strand of cDNA can be added to each individual RNA sample in the methods described herein. Such a template switching method is described in (Esumi et al., Neurosci Res 60 (4): 439-51 (2008)) and allows full length cDNA comprising the complete 5′ end of RNA to be synthesized. As the terminal transferase activity of reverse transcriptase typically causes 2-5 cytosines to be incorporated at the 3′ end of the first strand of cDNA synthesized from mRNA, the first strand of cDNA can include a plurality of cytosines, or cytosine analogues that base pair with guanosine, at its 3′ end (see U.S. Pat. No. 5,962,272). In one embodiment, the first strand of cDNA can include a 3′ portion comprising at least 2, at least 3, at least 4, at least 5 or 2, 3, 4, or 5 cytosines or cytosine analogues that base pair with guanosine. A non-limiting example of a cytosine analogue that base pairs with guanosine is 5-aminoallyl-2′-deoxycytidine.

In one embodiment, the template switch oligonucleotide can include a 3′ portion comprising a plurality of guanosines or guanosine analogues that base pair with cytosine. Non-limiting examples of guanosines or guanosine analogues useful in the methods described herein include, but are not limited to deoxyriboguanosine, riboguanosine, locked nucleic acid-guanosine, and peptide nucleic acid-guanosine. The guanosines can be ribonucleosides or locked nucleic acid monomers.

In a particular embodiment, the template switch oligonucleotide can include a 3′ portion including at least 2, at least 3, at least 4, at least 5, or 2, 3, 4, or 5, or 2-5 guanosines, or guanosine analogues that base pair with cytosine. The presence of a plurality of guanosines (or guanosine analogues that base pair with cytosine) allows the template switch oligonucleotide to anneal transiently to the exposed cytosines at the 3′ end of the first strand of cDNA. This causes the reverse transcriptase to switch template and continue to synthesis a strand complementary to the template switch oligonucleotide. In one embodiment, the 3′ end of the template switch oligonucleotide can be blocked, for example by a 3′ phosphate group, to prevent the template switch oligonucleotide from functioning as a primer during cDNA synthesis.

In another embodiment, the RNA is released from the cells by cell lysis. If the lysis is achieved partially by heating, then the cDNA synthesis primer and/or the template switch oligonucleotide can be added to each individual RNA sample during cell lysis, as this will aid hybridization of the oligonucleotides. In some embodiments, reverse transcriptase can be added after cell lysis to avoid denaturation of the enzyme.

In some embodiments of the disclosure, a tag can be incorporated into the cDNA during its synthesis. For example, the CDNA synthesis primer and/or the template switch oligonucleotide can include a tag, such as a particular nucleotide sequence, which can be at least 4, at least 5, at least 6, at least 7, at least 8, at least 9, at least 10, at least 15 or at least 20 nucleotides in length. For example, the tag can be a nucleotide sequence of 4-20 nucleotides in length, e.g., 4, 5, 6, 7, 8, 9, 10, 15 or 20 nucleotides in length. As the tag is present in the cDNA synthesis primer and/or the template switch oligonucleotide it will be incorporated into the cDNA during its synthesis and can therefore act as a “barcode” to identify the cDNA. Both the CDNA synthesis primer and the template switch oligonucleotide can include a tag. The cDNA synthesis primer and the template switch oligonucleotide can each include a different tag, such that the tagged cDNA sample comprises a combination of tags. Each cDNA sample generated by the above method can have a distinct tag, or a distinct combination of tags, such that once the tagged cDNA samples have been pooled, the tag can be used to identify which single cell from each cDNA sample originated. Thus, each cDNA sample can be linked to a single cell, even after the tagged cDNA samples have been pooled in the methods described herein.

Before the tagged cDNA samples are pooled, synthesis of CDNA can be stopped, for example by removing or inactivating the reverse transcriptase. This prevents cDNA synthesis by reverse transcription from continuing in the pooled samples. The tagged cDNA samples can optionally be purified before amplification, either before or after they are pooled.

If the RNA was not fragmented prior to conversion to CDNA, then the cDNA is fragmented and size selection is performed. CDNA can be fragmented with enzymes, sonication, or nebulizers. Fragmentation is followed by size selection, where either small sequences are removed or a tight range of sequence lengths are selected.

After the cDNA reaction, an end repair reaction is then performed with T4 Polynucleotide Kinase, rATP, and T4 DNA polymerase, dNTP, to form blunt ended double stranded templates. After end repair cleanup and size selection, an A-tailing reaction is performed with Klenow exo-, dNTP (e.g., dATP) (see FIG. 1) to facilitate ligation of an adapter. The adapter is formed by annealing two single-stranded oligonucleotides prepared by conventional automated oligonucleotide synthesis. The oligonucleotides are partially complementary such that the 3′ end of a first oligonucleotide is complementary to the 5′ end of a second oligonucleotide. The 5′ end of the first oligonucleotide and the 3′ end of second oligonucleotide are not complementary to each other. When the two strands are annealed, the resulting structure is double stranded at one end (the double-stranded region) and single stranded at the other end (the unmatched region) and is referred to herein as a “Y-shaped adapter”. The double-stranded region of the Y-shaped adapter may be blunt-ended or it may have an overhang. In the latter case, the overhang may be a 3′ overhang or a 5′ overhang, and may comprise a single nucleotide or more than one nucleotide. The Y-shaped adapter is phosphorylated at its 5′ end and the double-stranded portion of the duplex contains a single base 3′ overhang comprising a ‘T’ deoxynucleotide. The adapters are then ligated using T4 Ligase, rATP, to the ends of double stranded template molecules containing a single base 5′ overhand of an ‘A’ nucleotide.

The Y-shaped adapter is phosphorylated at its 5′ end and the double-stranded portion of the duplex contains a single base 3′ overhang comprising a ‘T’ deoxynucleotide (see FIG. 1). The adapters are then ligated using T4 Ligase, rATP, to the ends of double stranded template molecules containing a single base 5′ overhand of an ‘A’ nucleotide.

The library is generally formed by ligating adapter polynucleotide molecules to the 5′ and 3′ ends of one or more target polynucleotide duplexes (which may be of known, partially known or unknown sequence) to form adapter-target constructs and then carrying out PCR amplification to form a library of template polynucleotides. The library of template polynucleotides can then be sequenced using next generation sequencing. To save resources, multiple libraries can be pooled together and sequenced in the same run-a process known as multiplexing. During adapter ligation, unique index sequences, or “barcodes,” are added to each library. These barcodes are used to distinguish between the libraries during data analysis.

The adapters added onto the double stranded templates using the non-homologous end joining factors and methods of the disclosure typically comprise a double stranded region of complementary sequence and a single stranded region of sequence mismatch. In a particular embodiment, the adapters have a Y-shape, where the region of sequence mismatch causes the arms of the adapter to separate from each other. The “double-stranded region” of the adapter is a short double-stranded region, typically comprising 5 or more consecutive base pairs, formed by annealing of the two partially complementary polynucleotide strands. This term simply refers to a double-stranded region of nucleic acid in which the two strands are annealed and does not imply any particular structural conformation. In an alternate embodiment, the adapters, instead of having a Y-shape structure, are U-shaped, such that once the adapters are added to the ends of templates using the non-homologous end joining factors and methods of the disclosure form a continuous loop at the 5′ and 3′ ends of the templates. Accordingly, the resulting DNA library templates can be amplified using rolling circle amplification.

Generally, it is advantageous for the double-stranded region to be as short as possible without loss of function. By “function” in this context is meant that the double-stranded region forms a stable duplex under reaction conditions for the prokaryotic end joining and repair factors described herein, such that the two strands forming the adapter remain partially annealed during ligation of the adapter to a target molecule. It is not absolutely necessary for the double-stranded region to be stable under the conditions typically used in the annealing steps of PCR reactions.

In another embodiment, identical adapters are added to both ends of each template molecule, the target sequence in each adapter-target construct will be flanked by complementary sequences derived from the double-stranded region of the adapters. The longer the double-stranded region, and hence the complementary sequences derived therefrom in the adapter-target constructs, the greater the possibility that the adapter-target construct is able to fold back and base-pair to itself in these regions of internal self-complementarity under the annealing conditions used in PCR. Generally, it is preferred for the double-stranded region to be 20 or less, 15 or less, or 10 or less base pairs in length in order to reduce this effect. The stability of the double-stranded region may be increased, and hence its length potentially reduced, by the inclusion of non-natural nucleotides which exhibit stronger base-pairing than standard Watson-Crick base pairs.

It a particular embodiment, the two strands of the adapter to be 100% complementary in the double-stranded region. It will be appreciated, however, that one or more nucleotide mismatches may be tolerated within the double-stranded region, provided that the two strands are capable of forming a stable duplex under standard ligation conditions.

Alternatively, the adapters added onto the double stranded templates using the non-homologous end joining factors and methods of the disclosure comprise double stranded complementary sequences. The resulting adapter/template molecules can then be amplified by PCR to form the DNA library templates. In a further embodiment, a splint oligonucleotide can be used to join the ends of the DNA library templates to form a circle. An exonuclease is added to remove all remaining linear single-stranded and double-stranded DNA products. The result is a completed circular DNA template.

Adapters for use in the methods disclosed herein will generally include a double-stranded region adjacent to the “ligatable” end of the adapter, i.e., the end that is joined to a target polynucleotide using ligases or non-homologous end joining factors. The ligatable end of the adapter may be blunt or, in other embodiments, short 5′ or 3′ overhangs of one or more nucleotides may be present to facilitate/promote ligation. The 5′ terminal nucleotide at the ligatable end of the adapter should be phosphorylated to enable phosphodiester linkage to a 3′ hydroxyl group on the target polynucleotide.

The portions of the two strands forming the double-stranded region typically comprise at least 10, or at least 15, or at least 20 consecutive nucleotides on each strand. The lower limit on the length of the unmatched region will typically be determined by function, for example the need to provide a suitable sequence for binding of a primer for PCR and/or sequencing. Theoretically there is no upper limit on the length of the unmatched region, except that it general it is advantageous to minimize the overall length of the adapter, for example in order to facilitate separation of unbound adapters from adapter-target constructs following the ligation step. Therefore, it is preferred that the unmatched region should be less than 50, or less than 40, or less than 30, or less than 25 consecutive nucleotides in length on each strand.

The overall length of the two strands forming the adapter will typically in the range of from 25 to 100 nucleotides, more typically from 30 to 55 nucleotides.

The portions of the two strands forming the unmatched region should preferably be of similar length, although this is not absolutely essential, provided that the length of each portion is sufficient to fulfil its desired function (e.g., primer binding). It has been shown by experiment that the portions of the two strands forming the unmatched region may differ by up to 25 nucleotides without unduly affecting adapter function.

In a particular embodiment, the portions of the two polynucleotide strands forming the unmatched region will be completely mismatched, or 100% non-complementary. However, some sequence “matches”, i.e., a lesser degree of non-complementarity may be tolerated in this region without affecting function to a material extent. As aforesaid, the extent of sequence mismatching or non-complementarity is such that the two strands in the unmatched region remain in single-stranded form under annealing conditions as defined above.

The precise nucleotide sequence of the adapters is generally not material to the disclosure and may be selected by the user such that the desired sequence elements are ultimately included in the common sequences of the library of templates derived from the adapters, for example to provide binding sites for particular sets of universal amplification primers and/or sequencing primers (e.g., P7 or P5 primers). Additional sequence elements may be included, for example to provide binding sites for sequencing primers which will ultimately be used in sequencing of template molecules in the library, or products derived from amplification of the template library, for example on a solid support. The adapters may further include “bar code” sequences, which can be used to bar code template molecules derived from a particular source.

Although the precise nucleotide sequence of the adapter is generally non-limiting to the disclosure, the sequences of the individual strands in the unmatched region should be such that neither individual strand exhibits any internal self-complementarity which could lead to self-annealing, formation of hairpin structures, etc. under standard annealing conditions. Self-annealing of a strand in the unmatched region is to be avoided as it may prevent or reduce specific binding of an amplification primer to this strand.

The mismatched adapters are preferably formed from two strands of DNA, but may include mixtures of natural and non-natural nucleotides (e.g., one or more ribonucleotides) linked by a mixture of phosphodiester and non-phosphodiester backbone linkages. Other non-nucleotide modifications may be included such as, for example, biotin moieties, blocking groups and capture moieties for attachment to a solid surface, as discussed in further detail below.

The one or more “target polynucleotide duplexes” to which the adapters are ligated may be any polynucleotide molecules that can be used with additional methodologies, including amplification by solid-phase PCR, next generation sequencing, subcloning, etc. The target polynucleotide duplexes may originate in double-stranded DNA form (e.g., genomic DNA fragments) or may have originated in single-stranded form, as DNA or RNA, and been converted to dsDNA form prior to ligation. By way of example, mRNA molecules may be copied into double-stranded cDNAs suitable for use in the method of the disclosure using standard methodologies known in the art. The precise sequence of the target molecules is generally not material to the disclosure, and may be known or unknown. Modified DNA molecules including non-natural nucleotides and/or non-natural backbone linkages could serve as the target, provided that the modifications do not preclude adding on adapters, tagmentation of adapters to the DNA molecules, and/or copying by PCR.

As used herein, the term “tagmentation,” “tagment,” or “tagmenting” refers to transforming a nucleic acid, e.g., a DNA, into adaptor-modified templates such that the nucleic acid is modified to comprise 5′ and 3′ adapter molecules. This process often involves the modification of the nucleic acid by a transposome complex comprising transposase enzyme complexed with adaptors comprising transposon end sequence. Tagmentation results in the simultaneous fragmentation of the nucleic acid and ligation of the adaptors to the 5′ ends of both strands of duplex fragments. Following a purification step to remove the transposase enzyme, additional sequences can be added to the ends of the adapted fragments by PCR.

A “transposase” means an enzyme that is capable of forming a functional complex with a transposon end-containing composition (e.g., transposons, transposon ends, transposon end compositions) and catalyzing insertion or transposition of the transposon end-containing composition into the double-stranded target nucleic acid with which it is incubated, for example, in an in vitro transposition reaction. A transposase as presented herein can also include integrases from retrotransposons and retroviruses. Transposases, transposomes and transposome complexes are generally known to those of skill in the art, as exemplified by the disclosure of US Pat. Publ. No. 2010/0120098, the content of which is incorporated herein by reference in its entirety. Although many embodiments described herein refer to Tn5 transposase and/or hyperactive In5 transposase, it will be appreciated that any transposition system that is capable of inserting a transposon end with sufficient efficiency to 5′-tag and fragment a target nucleic acid for its intended purpose can be used in the present invention. In particular embodiments, a preferred transposition system is capable of inserting the transposon end in a random or in an almost random manner to 5′-tag and fragment the target nucleic acid.

As used herein, the term “transposition reaction” refers to a reaction wherein one or more transposons are inserted into target nucleic acids, e.g., at random sites or almost random sites. Essential components in a transposition reaction are a transposase and DNA oligonucleotides that exhibit the nucleotide sequences of a transposon, including the transferred transposon sequence and its complement (the non-transferred transposon end sequence) as well as other components needed to form a functional transposition or transposome complex. The DNA oligonucleotides can further comprise additional sequences (e.g., adaptor or primer sequences) as needed or desired. In some embodiments, the method provided herein is exemplified by employing a transposition complex formed by a hyperactive Tn5 transposase and a Tn5-type transposon end (Goryshin and Reznikoff, 1998, J. Biol. Chem., 273: 7367) or by a MuA transposase and a Mu transposon end comprising R1 and R2 end sequences (Mizuuchi, 1983, Cell, 35: 785; Savilahti et al., 1995, EMBO J., 14: 4893). However, any transposition system that is capable of inserting a transposon end in a random or in an almost random manner with sufficient efficiency to 5′-tag and fragment a target DNA for its intended purpose can be used in the present invention. Examples of transposition systems known in the art which can be used for the present methods include but are not limited to Staphylococcus aureus Tn552 (Colegio et al., 2001, J Bacterid., 183: 2384-8; Kirby et al., 2002, Mol Microbiol, 43: 173-86), TyI (Devine and Boeke, 1994, Nucleic Acids Res., 22: 3765-72 and International Patent Application No. WO 95/23875), Transposon Tn7 (Craig, 1996, Science. 271: 1512; Craig, 1996, Review in: Curr Top Microbiol Immunol, 204: 27-48), TnIO and ISlO (Kleckner et al., 1996, Curr Top Microbiol Immunol, 204: 49-82), Mariner transposase (Lampe et al., 1996, EMBO J., 15: 5470-9), Tci (Plasterk, 1996, Curr Top Microbiol Immunol, 204: 125-43), P Element (Gloor, 2004, Methods Mol Biol, 260: 97-114), TnJ (Ichikawa and Ohtsubo, 1990, J Biol Chem. 265: 18829-32), bacterial insertion sequences (Ohtsubo and Sekine, 1996, Curr. Top. Microbiol. Immunol. 204:1-26), retroviruses (Brown et al., 1989, Proc Natl Acad Sci USA, 86: 2525-9), and retrotransposon of yeast (Boeke and Corces, 1989, Annu Rev Microbiol. 43: 403-34). The method for inserting a transposon end into a target sequence can be carried out in vitro using any suitable transposon system for which a suitable in vitro transposition system is available or that can be developed based on knowledge in the art. In general, a suitable in vitro transposition system for use in the methods provided herein requires, at a minimum, a transposase enzyme of sufficient purity, sufficient concentration, and sufficient in vitro transposition activity and a transposon end with which the transposase forms a functional complex with the respective transposase that is capable of catalyzing the transposition reaction. Suitable transposase transposon end sequences that can be used in the invention include but are not limited to wild-type, derivative or mutant transposon end sequences that form a complex with a transposase chosen from among a wild-type, derivative or mutant form of the transposase.

As used herein, the term “transposome complex” refers to a transposase enzyme non-covalently bound to a double stranded nucleic acid. For example, the complex can be a transposase enzyme preincubated with double-stranded transposon DNA under conditions that support non-covalent complex formation. Double-stranded transposon DNA can include, without limitation, Tn5 DNA, a portion of Tn5 DNA, a transposon end composition, a mixture of transposon end compositions or other double-stranded DNAs capable of interacting with a transposase such as the hyperactive Tn5 transposase.

The term “transposon end” (TE) refers to a double-stranded nucleic acid, e.g., a double-stranded DNA that exhibits only the nucleotide sequences (the “transposon end sequences”) that are necessary to form the complex with the transposase or integrase enzyme that is functional in an in vitro transposition reaction. In some embodiments, a transposon end is capable of forming a functional complex with the transposase in a transposition reaction. As non-limiting examples, transposon ends can include the 19-bp outer end (“OE”) transposon end, inner end (“IE”) transposon end, or “mosaic end” (“ME”) transposon end recognized by a wild-type or mutant Tn5 transposase, or the R1 and R2 transposon end as set forth in the disclosure of US Pat. Publ. No. 2010/0120098, the content of which is incorporated herein by reference in its entirety. Transposon ends can include any nucleic acid or nucleic acid analogue suitable for forming a functional complex with the transposase or integrase enzyme in an in vitro transposition reaction. For example, the transposon end can include DNA, RNA, modified bases, non-natural bases, modified backbone, and can include nicks in one or both strands. Although the term “DNA” is sometimes used in the present disclosure in connection with the composition of transposon ends, it should be understood that any suitable nucleic acid or nucleic acid analogue can be utilized in a transposon end.

“Ligation” of adapters to 5′ and 3′ ends of each target polynucleotide involves joining of the two polynucleotide strands of the adapter to double-stranded target polynucleotide such that covalent linkages are formed between both strands of the two double-stranded molecules. In this context “joining” means covalent linkage of two polynucleotide strands which were not previously covalently linked. Preferably such “joining” will take place by formation of a phosphodiester linkage between the two polynucleotide strands but other means of covalent linkage (e.g., non-phosphodiester backbone linkages) may be used. However, the covalent linkages formed in the ligation reactions should allow for read-through of a polymerase, such that the resultant construct can be copied in a PCR reaction using primers which binding to sequences in the regions of the adapter-target construct that are derived from the adapter molecules.

The ligation reactions will typically be enzyme-catalyzed. In particular embodiment, the ligation reactions will be catalyzed by ligases or non-homologous end joining factors. Non-enzymatic ligation techniques (e.g., chemical ligation) may also be used, provided that the non-enzymatic ligation leads to the formation of a covalent linkage which allows read-through of a polymerase, such that the resultant construct can be copied by PCR.

The desired products of the ligation reaction are adapter-target constructs in which adapters are ligated at both ends of each target polynucleotide, given the structure adapter-target-adapter. Conditions of the ligation reaction should therefore be optimized to maximized the formation of this product, in preference to targets having an adapter at one end only.

The products of the tagmentation reaction or the ligation reaction may be subjected to purification steps in order to remove unbound adapter molecules before the adapter-target constructs are processed further. Any suitable technique may be used to remove excess unbound adapters, preferred examples of which will be described in further detail below.

The adapter-target constructs are then amplified by PCR, as described in further detail below. The products of such further PCR amplification may be collected to form a library of templates. In a certain embodiment, primers used for PCR amplification will anneal to different primer-binding sequences on opposite strands in the unmatched region of the adapter. Other embodiments may, however, be based on the use of a single type of amplification primer which anneals to a primer-binding sequence in the double-stranded region of the adapter.

As shown in FIG. 1, the new and improved method for depleting undesired sequences to form a template library provides for inclusion of one or more blocking oligonucleotides in the adapter-construct PCR amplification reaction. Thus, unlike in the standard RNA-Seq protocol, there is no need to treat the RNA sample to deplete the RNA sample of rRNA transcripts or to enrich the RNA sample for mRNA prior to conversion to cDNA. The simplicity of using the one or more blocking oligonucleotides of the disclosure to reduce non-desirable fragments is advantageous on automated library preparation systems, where reducing the number of reagents and steps are paramount for simple and robust workflows. The use of the one or more blocking oligonucleotides of the disclosure facilitates depletion of non-desirable fragments *after* library construction, enabling reduced hands-on time with labile RNA. Additionally, the use of PCR clamps can be combined with traditional rRNA depletion approaches on more challenging samples known to have biologically high amounts of rRNA, globin transcripts, or other non-desired transcripts.

It is generally advantageous for adapter-target constructs to be amplified by PCR in solution or on a solid support, to include regions of “different” sequence at their 5′ and 3′ ends, which are nevertheless are common to all template molecules in the library, especially if the amplification products are to be ultimately sequenced. For example, the presence of a common unique sequence at one end only of each template in the library can provide a binding site for a sequencing primer, enabling one strand of each template in the amplified form of the library to be sequenced in a single sequencing reaction using a single type of sequencing primer.

The conditions encountered during the annealing steps of a PCR reaction will be generally known to one skilled in the art, although the precise annealing conditions will vary from reaction to reaction (see Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al.). Typically, such conditions may comprise, but are not limited to, (following a denaturing step at a temperature of about 94° C. for about one minute) exposure to a temperature in the range of from 40° C. to 72° C. (preferably 50-68° C.) for a period of about 1 minute in standard PCR reaction buffer.

Inclusion of PCR amplification to form complementary copies of the adapter-target constructs is advantageous, for several reasons. Firstly, inclusion of the primer extension step, and subsequent PCR amplification, acts as an enrichment step to select for adapter-target constructs with adapters ligated at both ends, especially in the case of methods of the disclosure, as non-desired transcripts are not amplified in the PCR reaction. Only target constructs with adapters ligated at both ends provide effective templates for PCR using common or universal primers specific for primer-binding sequences in the adapters, hence it is advantageous to produce a template library comprising only double-ligated targets prior to PCR amplification.

Secondly, inclusion of PCR amplification, permits the length of the common sequences at the 5′ and 3′ ends of the target to be increased prior to sequencing. As outlined above, it is generally advantageous for the length of the adapter molecules to be kept as short as possible, to maximize the efficiency of ligation and subsequent removal of unbound adapters. However, for the purposes of sequencing it may be an advantage to have longer sequences common or “universal” sequences at the 5′ and 3′ ends of the templates to be amplified. Inclusion of PCR amplification means that the length of the common sequences at one (or both) ends of the polynucleotides in the template library can be increased after ligation by inclusion of additional sequence at the 5′ ends of the primers used for PCR amplification.

The template library prepared according to the methods disclosed herein can be used in any method of nucleic acid analysis, e.g., sequencing of the templates or amplification products thereof. Exemplary uses of the template libraries include, but are not limited to, providing templates for whole genome amplification, sequencing, subcloning, and PCR amplification (of either monotemplate or complex template libraries).

Template libraries prepared according to a method of the disclosure from a complex mixture of genomic DNA fragments representing a whole or substantially whole genome provide suitable templates for so-called “whole-genome” amplification. The term “whole-genome amplification” refers to a nucleic acid amplification reaction (e.g., PCR) in which the template to be amplified comprises a complex mixture of nucleic acid fragments representative of a whole (or substantially whole genome).

The library of templates prepared according to the methods described herein can be used for solid-phase nucleic acid amplification. The term “solid-phase amplification” as used herein refers to any nucleic acid amplification reaction carried out on or in association with a solid support such that all or a portion of the amplified products are immobilized on the solid support as they are formed. In particular, the term encompasses solid-phase polymerase chain reaction (solid-phase PCR), which is a reaction analogous to standard solution phase PCR, except that one or both of the forward and reverse amplification primers is/are immobilized on the solid support.

For “solid-phase” amplification methods, one amplification primer may be immobilized (the other primer usually being present in free solution). Alternatively, both the forward and the reverse primers may be immobilized. In practice, there will be a “plurality” of identical forward primers and/or a “plurality” of identical reverse primers immobilized on the solid support, since the PCR process requires an excess of primers to sustain amplification. References herein to forward and reverse primers are to be interpreted accordingly as encompassing a “plurality” of such primers unless the context indicates otherwise.

It is possible to carry out solid-phase amplification using only one type of primer, and such single-primer methods are encompassed within the scope of the disclosure. Other embodiments may use forward and reverse primers which contain identical template-specific sequences but which differ in some other structural features. For example, one type of primer may contain a non-nucleotide modification which is not present in the other. In other embodiments, the forward and reverse primers may contain template-specific portions of different sequence.

Amplification primers for solid-phase PCR are preferably immobilized by covalent attachment to the solid support at or near the 5′ end of the primer, leaving the template-specific portion of the primer free for annealing to its cognate template and the 3′ hydroxyl group free for primer extension. Any suitable covalent attachment means known in the art may be used for this purpose. The chosen attachment chemistry will depend on the nature of the solid support, and any derivatization or functionalization applied to it. The primer itself may include a moiety, which may be a non-nucleotide chemical modification, to facilitate attachment.

It is preferred to use the library of templates prepared according to a method disclosed herein to prepare clustered arrays of nucleic acid colonies by solid-phase PCR amplification. The terms “cluster” and “colony” are used interchangeably herein to refer to a discrete site on a solid support comprised of a plurality of identical immobilized nucleic acid strands and a plurality of identical immobilized complementary nucleic acid strands. The term “clustered array” refers to an array formed from such clusters or colonies. In this context the term “array” is not to be understood as requiring an ordered arrangement of clusters.

In a particular embodiment, the disclosure further provides methods of sequencing amplified nucleic acids generated by PCR amplification. Thus, the disclosure provides a method of nucleic acid sequencing comprising amplifying a library of nucleic acid templates using PCR as described above and carrying out a nucleic acid sequencing reaction to determine the sequence of the whole or a part of at least one amplified nucleic acid strand produced by PCR.

Sequencing can be carried out using any suitable “sequencing-by-synthesis” technique, wherein nucleotides are added successively to a free 3′ hydroxyl group, resulting in synthesis of a polynucleotide chain in the 5′ to 3′ direction. The nature of the nucleotide added is preferably determined after each nucleotide addition.

The initiation point for the sequencing reaction may be provided by annealing of a sequencing primer to a product of the whole genome or solid-phase amplification reaction. In this connection, one or both of the adapters added during formation of the template library may include a nucleotide sequence which permits annealing of a sequencing primer to amplified products derived by whole genome or solid-phase amplification of the template library.

The products of solid-phase amplification reactions wherein both forward and reverse amplification primers are covalently immobilized on the solid surface are so-called “bridged” structures formed by annealing of pairs of Immobilized polynucleotide strands and immobilized complementary strands, both strands being attached to the solid support (e.g., a flowcell) at the 5′ end. Arrays comprised of such bridged structures provide inefficient templates for nucleic acid sequencing, since hybridization of a conventional sequencing primer to one of the immobilized strands is not favored compared to annealing of this strand to its immobilized complementary strand under standard conditions for hybridization.

In order to provide more suitable templates for nucleic acid sequencing it is preferred to remove substantially all or at least a portion of one of the immobilized strands in the “bridged” structure in order to generate a template which is at least partially single-stranded. The portion of the template which is single-stranded will thus be available for hybridization to a sequencing primer. The process of removing all or a portion of one immobilized strand in a “bridged” double-stranded nucleic acid structure may be referred to herein as “linearization”.

Bridged template structures may be linearized by cleavage of one or both strands with a restriction endonuclease or by cleavage of one strand with a nicking endonuclease. Other methods of cleavage can be used as an alternative to restriction enzymes or nicking enzymes, including inter alia chemical cleavage (e.g., cleavage of a diol linkage with periodate), cleavage of abasic sites by cleavage with endonuclease, or by exposure to heat or alkali, cleavage of ribonucleotides incorporated into amplification products otherwise comprised of deoxyribonucleotides, photochemical cleavage or cleavage of a peptide linker.

It will be appreciated that a linearization step may not be essential if the solid-phase amplification reaction is performed with only one primer covalently immobilized and the other in free solution.

In order to generate a linearized template suitable for sequencing it is necessary to remove “unequal” amounts of the complementary strands in the bridged structure formed by amplification so as to leave behind a linearized template for sequencing which is fully or partially single stranded. Most preferably one strand of the bridged structure is substantially or completely removed.

Following the cleavage step, regardless of the method used for cleavage, the product of the cleavage reaction may be subjected to denaturing conditions in order to remove the portion (s) of the cleaved strand (s) that are not attached to the solid support. Suitable denaturing conditions will be apparent to the skilled reader with reference to standard molecular biology protocols (Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al.).

Denaturation (and subsequent re-annealing of the cleaved strands) results in the production of a sequencing template which is partially or substantially single-stranded. A sequencing reaction may then be initiated by hybridization of a sequencing primer to the single-stranded portion of the template.

Thus, the nucleic acid sequencing reaction may comprise hybridizing a sequencing primer to a single-stranded region of a linearized amplification product, sequentially incorporating one or more nucleotides into a polynucleotide strand complementary to the region of amplified template strand to be sequenced, identifying the base present in one or more of the incorporated nucleotide (s) and thereby determining the sequence of a region of the template strand.

One preferred sequencing method which can be used in accordance with the disclosure relies on the use of modified nucleotides that can act as chain terminators. Once the modified nucleotide has been incorporated into the growing polynucleotide chain complementary to the region of the template being sequenced there is no free 3′—OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached a different label, known to correspond to the particular base, to facilitate discrimination between the bases added at each incorporation step. Alternatively, a separate reaction may be carried out containing each of the modified nucleotides separately.

The modified nucleotides may carry a label to facilitate their detection. Preferably this is a fluorescent label. Each nucleotide type may carry a different fluorescent label. However, the detectable label need not be a fluorescent label. Any label can be used which allows the detection of an incorporated nucleotide.

One method for detecting fluorescently labelled nucleotides comprises using laser light of a wavelength specific for the labelled nucleotides, or the use of other suitable sources of illumination. The fluorescence from the label on the nucleotide may be detected by a CCD camera or other suitable detection means.

The disclosure is not intended to be limited to use of the sequencing method outlined above, as essentially any sequencing methodology which relies on successive incorporation of nucleotides into a polynucleotide chain can be used. Suitable alternative techniques include, for example, Pyrosequencing™, FISSEQ (fluorescent in situ sequencing), MPSS (massively parallel signature sequencing) and sequencing by ligation-based methods.

The target polynucleotide to be sequenced using the method of the disclosure may be any polynucleotide that it is desired to sequence. Using the template library preparation method described in detail herein it is possible to prepare template libraries starting from essentially any double or single-stranded target polynucleotide of known, unknown or partially known sequence. With the use of clustered arrays prepared by solid-phase amplification it is possible to sequence multiple targets of the same or different sequence in parallel.

Various non-limiting specific embodiments of the method of the disclosure will now be described in further detail with reference to the accompanying drawings. Features described as being preferred in relation to one specific embodiment of the disclosure apply mutatis mutandis to other specific embodiments of the disclosure unless stated otherwise.

FIG. 1, as described in detail above, provides RNA-Seq technology for the generation of a sequencing library from an RNA sample. Unlike with the traditional RNA workflow, the workflow enabled by addition of one or more blocking oligonucleotides specific to non-desirable rRNA fragments does not require a lengthy 1-to-2-hour depletion of rRNA prior to conversion of the RNA into CDNA, as is the case with on-market technologies. This enables faster workflow times and, in some implementations, easier automation due to the reduced needs for various reagents.

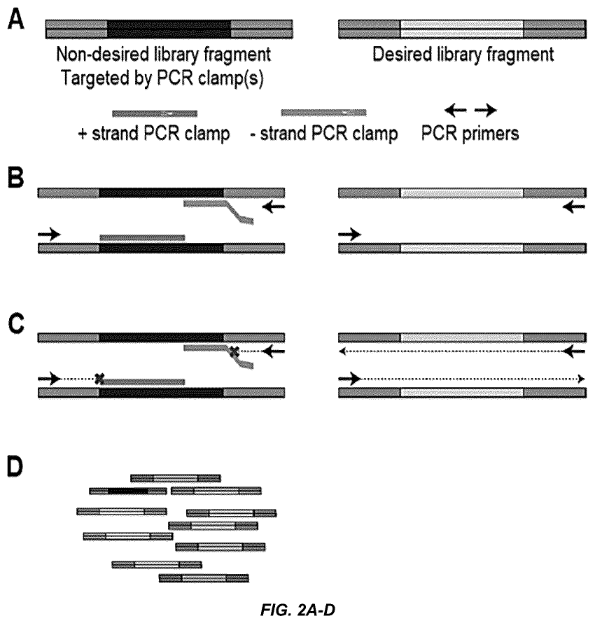

FIG. 2 provides an illustration and overview of an exemplary method of disclosure. As shown, PCR clamps selectively block amplification of targeted, non-desired library fragments (see FIG. 2A). Following denaturation of libraries in the initial heat-denaturation step of PCR, amplification primers bind to the end of library fragments. PCR clamps, designed to be complementary to non-desirable fragments, also hybridize to select library fragments (see FIG. 2B). The thermostable polymerase can extend the primers and copy desired library fragments. However, because typical thermostable polymerases used in PCR lack 5′ to 3′ exonuclease and strand displacement activities, the PCR clamp effectively blocks copying of the non-desired fragment (see FIG. 2C). After several cycles of PCR, the desired library fragments have been amplified exponentially, while amplification of the non-desired fragments has been suppressed. The result is a final amplified library with reduced representation of the non-desired library fragments (see FIG. 2D). The method of the disclosure was found to work well with Kapa HiFi polymerase due to its lack of 5′→3′ exonuclease activity and strand displacement.

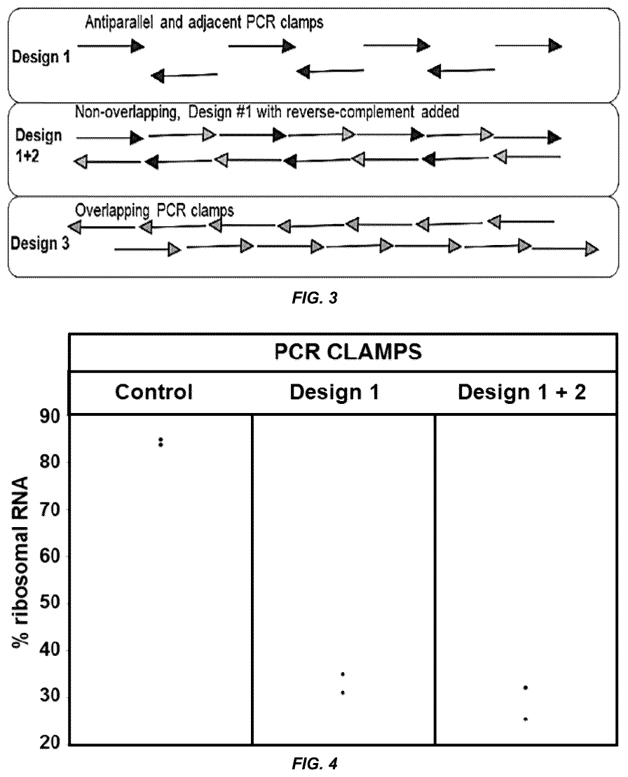

FIG. 3 provides various designs of pools of blocking oligonucleotides (i.e., PCR clamps) to deplete non-desired transcripts from a template library. Design 1 provides for a pool of antiparallel and adjacent PCR clamps. Design 1+2 provides for the same pool of PCR clamps of Design 1 but reverse-complement PCR clamps have been added to the pool. Design 3 provides for antiparallel overlapping PCR clamps.

FIG. 4 shows that the pool of PCR clamps of Design 1 and the pool of PCR clamps of Design1_2 reduced the percentage of rRNA transcripts from 80% to 30% in an RNA-seq protocol using non-depleted RNA. No additional workup steps were required.

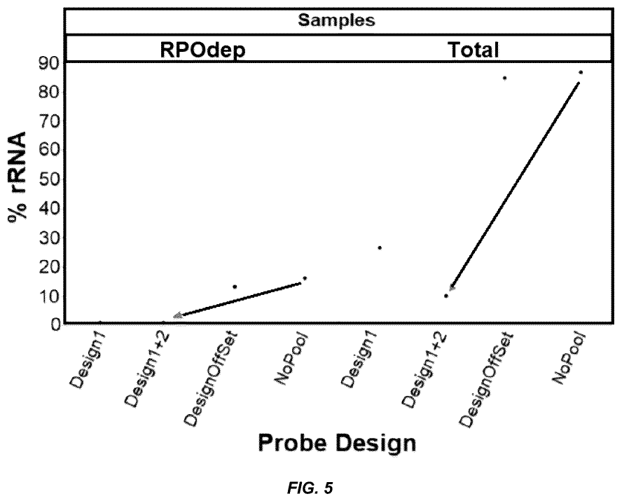

FIG. 5 shows that the pool of PCR clamps of Design 1 and the pool of PCR clamps of Design1_2 further reduced the percentage of rRNA transcripts from 208 to 18 in an RNA-seq protocol using an RPO depleted RNA sample (Left Panel). The RPO depleted RNA sample is enriched with library fragments of interest though some unwanted ribosomal rRNA is still observed (20%). (RPO=RNA Pan-Cancer Oligos (i.e., oligos from Illumina™ TruSight RNA Pan-Cancer product)). Further, the pool of PCR clamps of Design 1 and the pool of PCR clamps of Design1_2 were able to deplete rRNA transcripts in a non-depleted RNA sample to a comparable level as the RPO depleted RNA sample (Right Panel). Design 3 (DesignOffSet) was unable to deplete samples of rRNA transcripts. It is postulated that the PCR clamps were priming off each other to form secondary structures of rRNA artefacts.

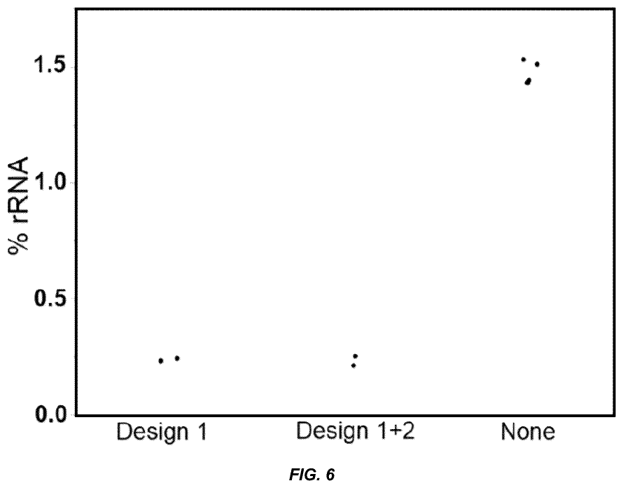

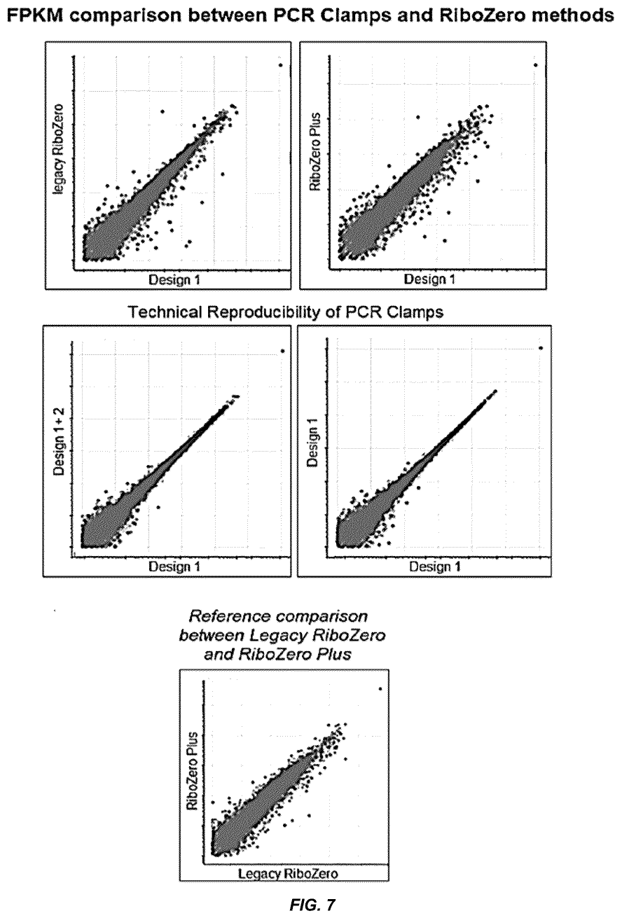

FIG. 6 shows that the pool of PCR clamps of Design 1 and the pool of PCR clamps of Design1_2 further reduced the percentage of rRNA transcripts from 1.5% to 0.25% in an RNA-seq protocol using an mRNA selected sample.

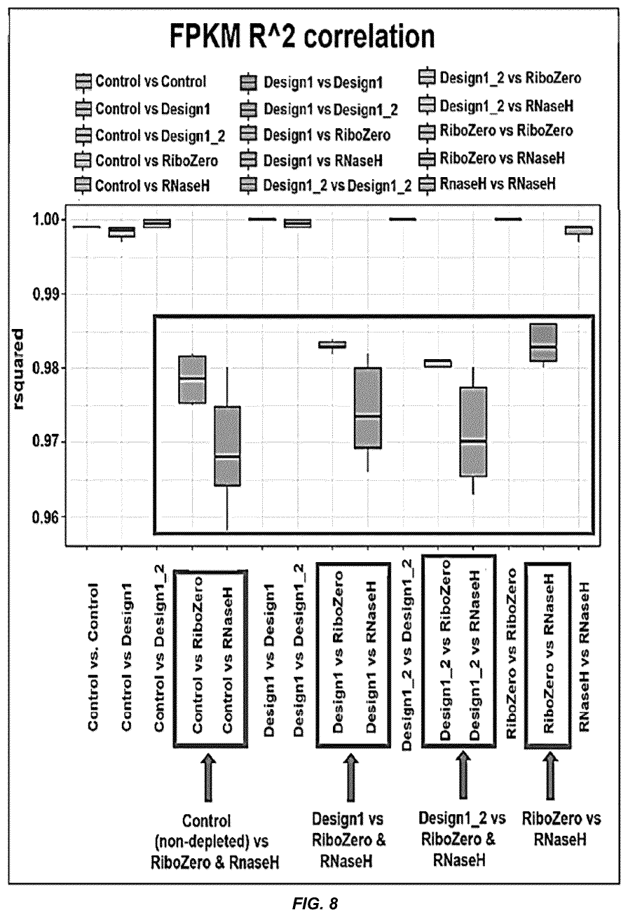

FIG. 8 shows that samples depleted by the PCR clamps of Design 1 or the PCR clamps of Design1_2 exhibited a high level of gene expression as by the Fragments Per Kilobase of transcript per Million mapped reads (FPKM) exhibiting a value of >0.95 which was equivalent to other depletion methods.

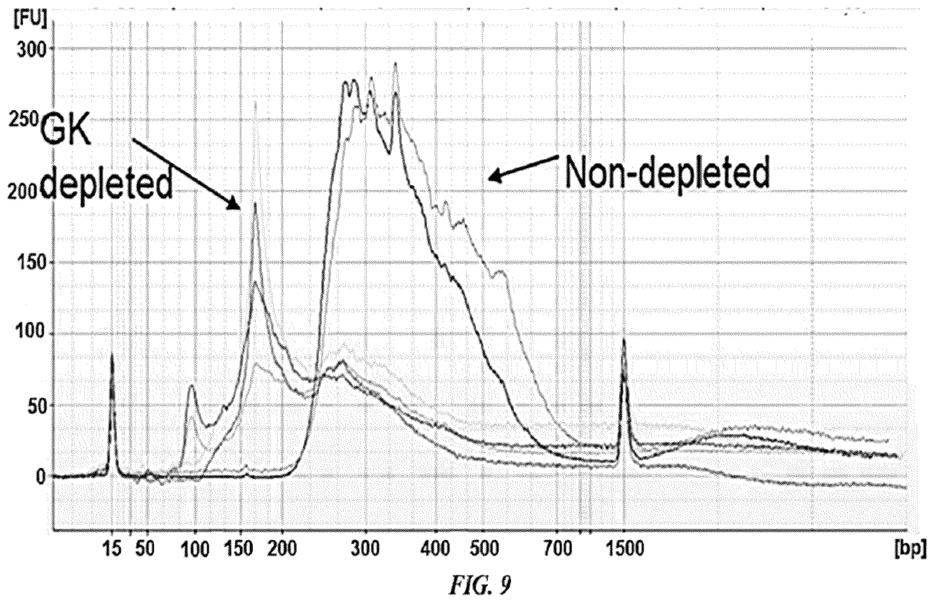

FIG. 9 provides a tracing showing that rRNA transcripts were greatly reduced in samples depleted of rRNA using blocking oligonucleotides v. non-depleted samples.

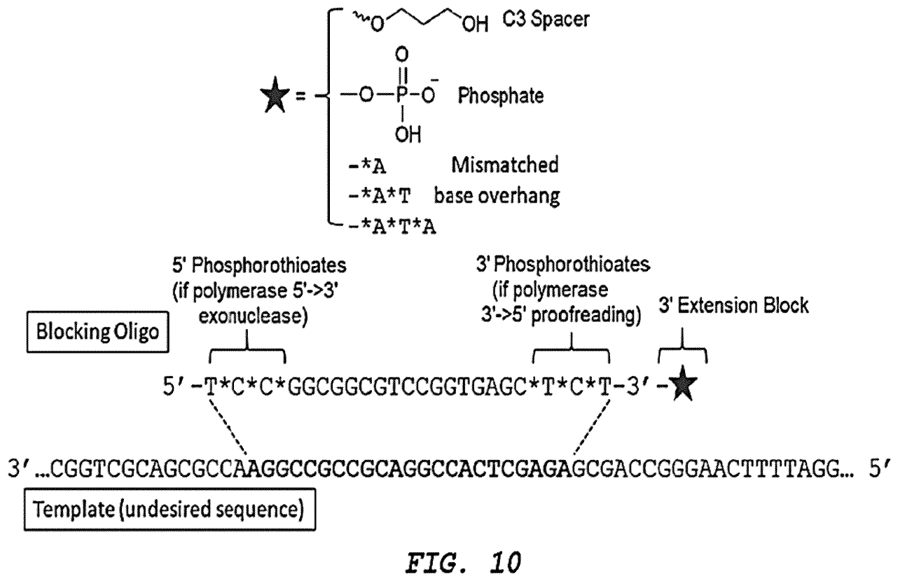

FIG. 10 presents an exemplary blocking oligonucleotide of the disclosure. The blocking oligonucleotide is designed to hybridize with internal (i.e., not overlapping primer binding sites) regions of the target fragment (s). Because most DNA polymerases used in PCR lack significant strand-displacement activity, the presence of a sufficiently strongly-bound blocking oligonucleotide should physically hinder progression of the polymerase and prevent synthesis of a full-length amplicon. Considerations for the blocking nucleotide include, but are not limited to:
(1) Having a melting temperature (Tm) higher than the temperature of the extension step in the PCR reaction. This ensures that the blocking oligonucleotide remains bound through the PCR extension step.
(2) The blocking oligo nucleotide can comprise a 3′-block on its 3′ terminus to prevent polymerase extension. This 3′-block prevents the blocking oligonucleotide from acting as a primer and generating unwanted PCR side products. Several methods can be used to achieve this, including 3′ spacer modifications (e.g., C3), 3′ inverted bases, 3′ phosphorylation, 3′ dideoxy bases, or 3′ non-complementary overhanging bases.
(3) If a proofreading DNA polymerase (i.e., a polymerase with strong 3′->5′ exonuclease activity) is used in the PCR reaction, the blocking oligo should be resistant to exonuclease activity at the 3′ end to prevent degradation. This can be achieved by the blocking oligonucleotide comprising 1 or more phosphorthioate linkages at the 3′ end of the blocking oligonucleotide.
(4) If a polymerase with strong 5′->3′ exonuclease activity (e.g., Taq DNA polymerase) is used, the blocking oligo should be resistant to exonuclease degradation at its 5′ end. This can be achieved by the blocking oligonucleotide comprising 1 or more phosphorthioate linkages at the 5′ end of the blocking oligonucleotide.

Due to the sequence dependence for Tm, the length of oligo needed to achieve consideration (1) can be prohibitively long, particularly for AT-rich sequences. Additional oligo modifications, such as Locked Nucleic Acid (LNA) bases or Peptide Nucleic Acid (PNA) linkages can be used in this circumstance to raise the Tm of the blocking oligonucleotide without changing the length or sequence of the blocking oligonucleotide.

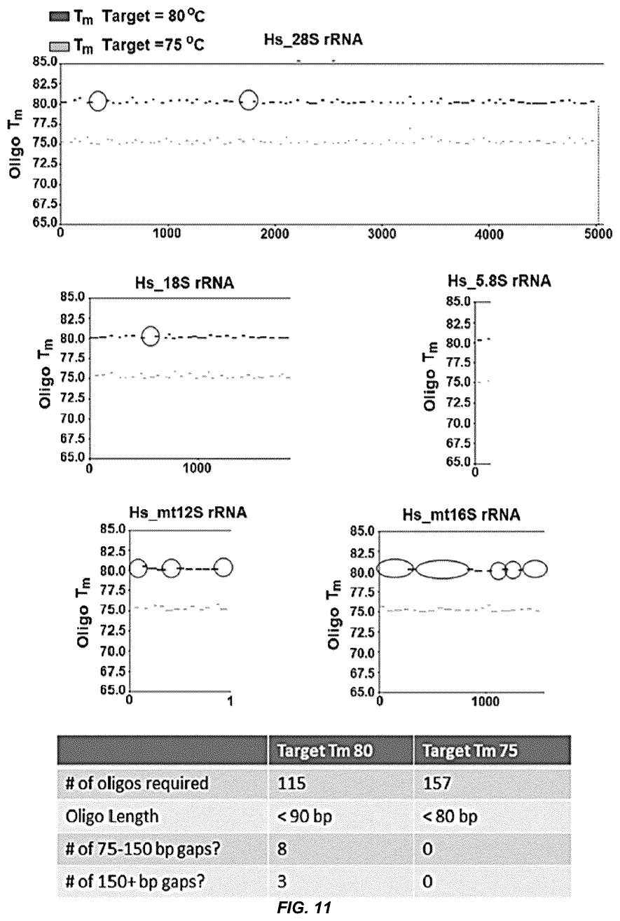

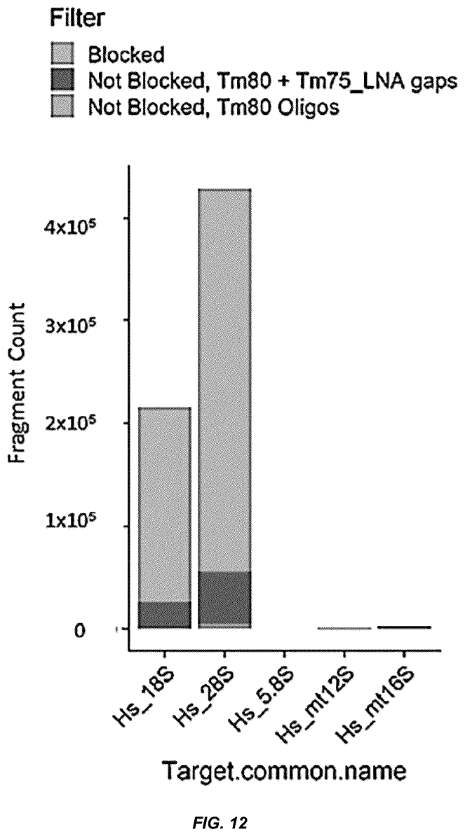

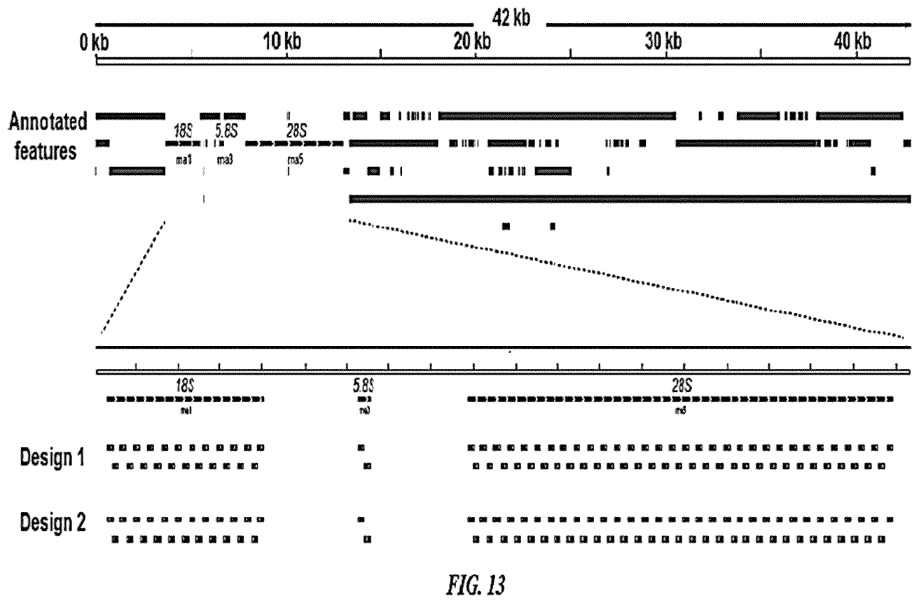

FIG. 11-12 demonstrate the use of blocking oligonucleotides to deplete ribosomal sequences from RNA-seq libraries. A pool of blocking oligos can be designed such that the majority of potential library fragments from each of the five major rRNA sequences (18S, 28S, 5S, mitochondrial 12S, and mitochondrial 16S) are targeted by one or more blocking oligonucleotides. The pool of blocking oligos can then be added to the sample during the PCR amplification step of library preparation, resulting in specific depletion of rRNA amplicons in the final library.

In addition to the general blocking oligonucleotide considerations outlined above, several additional parameters need to be considered for rRNA blocking oligonucleotide pool design:
(1) The length of blocking oligonucleotides should be minimized as much as possible while maintaining the target Tm. This allows the largest number of possible rRNA library fragments to be covered by an end-to-end match with a blocking oligo.
(2) Blocking oligonucleotide spacing should be chosen to minimize the number of gaps larger than the insert size of the target library.
(3) Blocking oligonucleotides may need to be designed to target both the sense and antisense strands of the targeted rRNA fragments.

A computational strategy was implemented to design a pool of rRNA blocking oligos for use with human RNA-seq libraries, comprising the following steps:
(1) Starting from the 5′ end of each rRNA sequence, a window of 90 bp (approximately 0.5× the average insert size for RNA libraries) was designated and scanned for oligos with a Tm above 80° C. Oligo length was initially set to 15 bp, and increased iteratively until either (a) an oligo with the desired Tm was found or (b) oligo length exceeded 90 bp.
(2) Once an oligo is identified within the window, a new 90 bp window is set beginning at the 3′ end of the oligo and the search procedure from step (1) is repeated. If no oligo is found within a given window, a new window is set beginning at the 3′ end of the previous window.
(3) Steps (1) and (2) are repeated until the end of the sequence is reached.

Using this approach, a set of blocking oligos were designed that covered almost the entire length of the 5 human rRNAs (see FIGS. 11 and 12) with only 11 gaps greater than 90 bp across all sequences. Simulations using an un-depleted RNA seq library (i.e., consisting mostly of rRNAs) showed that nearly 90% of rRNA library fragments will be targeted for depletion by one or more of the blocking oligonucleotides from the designed pool. This suggests that the blocking oligonucleotide approach described herein could give comparable depletion efficiency to commercially available rRNA-depletion kits (e.g., ˜95% depletion for RiboMinus) with a greatly simplified workflow and better performance on low-input RNA samples. This approach to pool design could also be applied to other NGS methods where contamination by abundant sequences is problematic, such as detection of rare somatic mutations, NIPT, metagenomics, or pathogen detection.

Accordingly, in the studies presented herein, it was shown that pools of blocking oligonucleotides (i.e., PCR clamps) selectively prevented PCR amplification of undesired library fragments. The depletion of undesired transcripts from a library requires no extra work up steps by the user, and only one or more blocking polynucleotides need to be added to the PCR amplification reaction. The studies clearly demonstrate that use of one can selectively reduce rRNA content in amplified RNA-Seq libraries by using the one or more blocking oligonucleotides (i.e., PCR clamps) of the disclosure. Further, in samples treated with rRNA depletion agents (RPO treated) and mRNA selected samples, the use of one or more blocking oligonucleotides significantly further reduced rRNA content in these samples. For example, in RPO treated samples, the use of one or more blocking oligonucleotides (i.e., PCR clamps) of the disclosure reduced rRNA content to <1% rRNA from ˜10-15%.

In comparison to other rRNA depletion techniques, the compositions, methods and kits of the disclosure provide for faster preparation of depleted RNA libraries using an RNA-Seq workflow. Moreover, the compositions, methods and kits of the disclosure depleted rRNA content from 80% to 30% which was comparable to existing rRNA depletion techniques. The compositions, methods and kits of the disclosure are fully compatible with existing rRNA depletion techniques and can be used with said techniques to further reduce rRNA content down to barely detectable levels. There were few observed off-target effects, and the compositions, methods and kits of the disclosure maintained a high correlation of gene level expression that was comparable to Ribozero and RNase H depletion methods. The number of cycles in the PCR reaction is correlative to the level of reduction of undesirable transcripts in the resulting library. In other words, the higher the PCR cycle number the greater the reduction of undesirable transcripts in the resulting library.

It should be noted that the studies were conducted with blocking oligonucleotides (i.e., PCR clamps) where no 3′-blocks were utilized. It would be expected that blocking oligonucleotides can provide further improvements in depleting samples of undesired transcripts and likely greatly reduce formation of concatemers in overlapping blocking nucleotides (Design 3). In cases where the Tm of the blocking nucleotides needs to be increased without increasing the length of the blocking oligonucleotide, modified bases, such as LNA or PNA may be used.

While the studies were geared to depleting rRNA transcripts from a total RNA sample, it is expected that the methods, compositions, and kits of the disclosure are generally applicable for reducing undesirable transcripts in a library preparation. For examples, one or more blocking oligonucleotides can be used to reduce undesirable mtDNA in ATAC-Seq preparations; or to reduce host transcripts for epidemiology samples.

The disclosure further provides for kits comprising one or more blocking oligonucleotides disclosed herein. The kits can be tailored for use in particular applications. For example, the kits can be directed to the use of the one or more blocking oligonucleotides in preparing libraries of template polynucleotides using the methods of the disclosure. Such kits can comprise at least a supply of adapters as defined herein, plus a supply of at least one amplification primer which is capable of annealing to the adapter and priming synthesis of an extension product, which extension product would include any target sequence ligated to the adapter when the adapter is in use. The structure and properties of amplification primers will be well known to those skilled in the art. Suitable primers of appropriate nucleotide sequence for use with the adapters included in the kit can be readily prepared using standard automated nucleic acid synthesis equipment and reagents in routine use in the art. The kit may include as supply of one single type of primer or separate supplies (or even a mixture) of two different primers, for example a pair of PCR primers suitable for PCR amplification of templates modified with the mismatched adapter in solution phase and/or on a suitable solid support (i.e., solid-phase PCR).

Adapters, PCR primers, and one or more blocking oligonucleotides may be supplied in the kits ready for use, or more preferably as concentrates-requiring dilution before use, or even in a lyophilized or dried form requiring reconstitution prior to use. If required, the kits may further include a supply of a suitable diluent for dilution or reconstitution of the primers. Optionally, the kits may further comprise supplies of reagents, buffers, enzymes, dNTPs etc. for use in carrying out PCR amplification. Further components which may optionally be supplied in the kit include “universal” sequencing primers suitable for sequencing templates prepared using the adapters and primers.

The disclosure further provides that the methods and compositions described herein can be further defined by the following aspects (aspects 1 to 43):

1. A method to selectively deplete non-desirable fragments from amplified DNA or cDNA libraries by using one or more blocking oligonucleotides, comprising:
amplifying in a polymerase chain reaction (PCR) reaction, a plurality of library fragments comprising a double stranded template sequence including adapter sequences, wherein a portion of the fragments comprise non-desirable fragments that are not to be analyzed;
wherein the PCR reaction comprises a plurality of fragments, a polymerase, dNTPS, PCR primers, and one or more blocking oligonucleotides, wherein the one or more blocking oligonucleotides comprise (i) and/or (ii), and (iii):
(i) at the 5′ terminus, one or more nucleotides that comprise a phosphorothioate linkage; and/or
(ii) at the 3′terminus, one or more nucleotides that comprise a phosphorothioate linkage; and
(iii) a 3′-block that prevent polymerase extension on the 3′ terminus of the blocking oligonucleotide;
wherein the one or more blocking primers bind to the template sequences of non-desired fragments, thereby blocking amplification of the non-desired fragments by PCR.

2. The method of aspect 1, wherein the one or more of the blocking oligonucleotides are from 15 nt to 100 nt in length, preferably wherein the blocking nucleotides are from 15 nt to 80 nt, 15 nt to 70 nt, 15 nt to 60 nt, 15 nt to 50 nt, 15 nt to 40 nt, 15 nt to 30 nt, 17 nt to 30 nt, or 20 nt to 30 nt in length.

3. The method of aspect 1 or aspect 2, wherein if the polymerase has 5′ to 3′ exonuclease activity, then the one or more of the blocking oligonucleotides comprise at the 5′ terminus, 1 to 5 nucleotides that comprise a phosphorothioate linkage, preferably wherein the 5′ terminus comprises 2 to 5, 3 to 5, 4 to 5, 2 to 4, or 2 to 3 nucleotides that comprises a phosphorothioate linkage.

4. The method of any one of the previous aspects, wherein if the polymerase has 3′ to 5′ proofreading activity, then the one or more of the blocking oligonucleotides comprise at the 3′ terminus, 1 to 5 nucleotides that comprise a phosphorothioate linkage, preferably wherein the 3′ terminus comprises 2 to 5, 3 to 5, 4 to 5, 2 to 4, or 2 to 3 nucleotides that comprises a phosphorothioate linkage.

5. The method of any one of the previous aspects, wherein the one or more blocking oligonucleotides comprise (i), (ii), and (iii):
(i) at the 5′ terminus, 2 to 5 nucleotides that comprise a phosphorothioate linkage, preferably wherein the 5′ terminus comprises 2 to 5, 3 to 5, 4 to 5, 2 to 4, or 2 to 3 nucleotides that comprises a phosphorothioate linkage.
   ; and/or
(ii) at the 3′terminus, 2 to 5 nucleotides that comprise a phosphorothioate linkage, preferably wherein the 3′ terminus comprises 2 to 5, 3 to 5, 4 to 5, 2 to 4, or 2 to 3 nucleotides that comprises a phosphorothioate linkage.
   ; and
(iii) a 3′-block that prevent polymerase extension on the 3′ terminus of the blocking oligonucleotide.

6. The method of any one of the previous aspects, wherein the 3′-block is selected from a C₃-spacer, 3′ inverted bases, 3′ phosphorylation, 3′ dideoxy bases or 3′ non-complementary overhanging bases, preferably where the 3′-block is a C₃-spacer.

7. The method of any one of the previous aspects, wherein the amplified libraries comprise template sequences from cDNA.

8. The method of any one of the previous aspects, wherein the amplified libraries comprise template sequences from gDNA.

9. The method of any one of the previous aspects, wherein the adapter sequences are from Y-shaped adapters that have been ligated to each end of a template sequence.

10. The method of any one of the previous aspects, wherein the one or more blocking oligonucleotides bind to template sequences from rRNAs and/or globin.

11. The method of any one of the previous aspects, wherein the one or more blocking oligonucleotides comprise a pool of blocking oligonucleotides that bind to template sequences from 18S rRNA, 5.8S rRNA, and/or 28S RNA.

12. The method of any one of the previous aspects, wherein the one or more of the blocking oligonucleotides bind to template sequences from mtDNA.

13. The method of any one of the previous aspects, wherein the amplified DNA or cDNA libraries are analyzed by using next generation sequencing.

14. The method of any one of the previous aspects, wherein the PCR amplification step is preceded by the following steps: obtaining an RNA sample;
fragmenting the RNA, preferably by sonification, use of enzymes, heat alone, or exposure to divalent cations at an elevated temperature;
reverse transcribing the RNA fragments to cDNA;
blunt ending the cDNA and adding an A nucleotide to the 3′ end of the blunt ended cDNA; and
ligating the A-tailed cDNA with adapters comprising a non-complemented T nucleotide at the 3′ end.

15. The method of aspect 14, wherein prior to reverse transcribing the RNA fragments to cDNA, the RNA sample is treated to deplete rRNA sequences from the RNA sample.

16. The method of any one of aspects 1 to 13, wherein the PCR amplification step is preceded by tagmentation reaction step to generate a plurality of library fragments comprising a double stranded template sequence including adapter sequences.

17. A method to selectively deplete non-desirable fragments from amplified DNA or cDNA libraries by using one or more blocking oligonucleotides, comprising:
amplifying in a polymerase chain reaction (PCR) reaction, a plurality of library fragments comprising a double stranded template sequence that has been ligated to adapter sequences, wherein a portion of the fragments comprise non-desirable fragments that contain template sequences that are not to be analyzed;
wherein the PCR reaction comprises a plurality of fragments, a polymerase, dNTPS, PCR primers, and a pool of blocking oligonucleotides, wherein a portion of the pool of the blocking oligonucleotides bind to each strand of a template sequence of a non-desired fragment;
wherein the one or more blocking primers bind to the template sequences of non-desired fragments, thereby blocking amplification of the non-desired fragments by PCR.

18. The method of aspect 17, wherein the pool of blocking oligonucleotides are from 15 nt to 100 nt in length, preferably wherein the blocking nucleotides are from 15 nt to 80 nt, 15 nt to 70 nt, 15 nt to 60 nt, 15 nt to 50 nt, 15 nt to 40 nt, 15 nt to 30 nt, 17 nt to 30 nt, or 20 nt to 30 nt in length.

19. The method of aspect 17, wherein the pool of blocking oligonucleotides comprise blocking oligonucleotides which bind to the strands of the template in a nonoverlapping and adjacent manner, preferably in the manner of Design 1 of FIG. 3.

20. The method of aspect 19, wherein the pool of blocking oligonucleotides comprise blocking oligonucleotides that are reverse-complement to other blocking oligonucleotides, preferably in the manner of Design 1+2 of FIG. 3.

21. The method of any one of aspects 17 to 20, wherein the pool of blocking oligonucleotides comprise (i) and/or (ii), and (iii):
(i) at the 5′ terminus, one or more nucleotides that comprise a phosphorothioate linkage; and/or
(ii) at the 3′terminus, one or more nucleotides that comprise a phosphorothioate linkage; and
(iii) a 3′-block that prevent polymerase extension on the 3′ terminus of the blocking oligonucleotide.

22. The method of aspect 21, wherein if the polymerase has 5′ to 3′ exonuclease activity, then the one or more of the blocking oligonucleotides comprise at the 5′ terminus, 1 to 5 nucleotides that comprise a phosphorothioate linkage.

23. The method of aspect 21, wherein if the polymerase has 3′ to 5′ proofreading activity, then the one or more of the blocking oligonucleotides comprise at the 3′ terminus, 1 to 5 nucleotides that comprise a phosphorothioate linkage.

24. The method of aspect 21, wherein the one or more blocking oligonucleotides comprise (i), (ii), and (iii):
(i) at the 5′ terminus, 2 to 5 nucleotides that comprise a phosphorothioate linkage;
(ii) at the 3′terminus, 2 to 5 nucleotides that comprise a phosphorothioate linkage; and
(iii) a 3′-block that prevent polymerase extension on the 3′ terminus of the blocking oligonucleotide.

25. The method of any one of aspects 21 to 24, wherein the 3′-block is selected from a C₃-spacer, 3′ inverted bases, 3′ phosphorylation, 3′ dideoxy bases or 3′ non-complementary overhanging bases.

26. The method of any one of aspects 17 to 25, wherein the amplified libraries comprise template sequences from cDNA.

27. The method of any one of aspects 17 to 25, wherein the amplified libraries comprise template sequences from gDNA.

28. The method of any one of aspects 17 to 27, wherein the adapter sequences are from Y-shaped adapters that have been ligated to each end of a template sequence.

29. The method of any one of aspects 17 to 28, wherein the pool of blocking oligonucleotides bind to template sequences from rRNAs and/or globin.

30. The method of any one of aspects 17 to 29, wherein the pool of blocking oligonucleotides bind to template sequences from 18S rRNA, 5.8S rRNA, and/or 28S RNA.

31. The method of any one of aspects 17 to 30, wherein the pool of blocking of blocking oligonucleotides bind to template sequences from mtDNA.

32. The method of any one of aspects 17 to 31, wherein the amplified DNA or cDNA libraries are analyzed by using next generation sequencing.

33. The method of any one of aspects 17 to 32, wherein the PCR amplification step is preceded by the following steps:
obtaining an RNA sample;
fragmenting the RNA, preferably by sonification, use of enzymes, heat alone, or exposure to divalent cations at an elevated temperature;
reverse transcribing the RNA fragments to cDNA;
blunt ending the cDNA and adding an A nucleotide to the 3′ end of the blunt ended cDNA; and
ligating the A-tailed cDNA with adapters comprising a non-complemented T nucleotide at the 3′ end.

34. The method of aspect 33, wherein prior to reverse transcribing the RNA fragments to cDNA, the RNA sample is treated to deplete rRNA sequences from the RNA sample.

35. The method of any one of aspects 17 to 34, wherein the PCR amplification step is preceded by tagmentation reaction step to generate a plurality of library fragments comprising a double stranded template sequence including adapter sequences.

36. An RNA-Seq based library preparation kit comprising one or more blocking oligonucleotides, wherein the one or more blocking oligonucleotides comprise (i) and/or (ii), and (iii):
(i) at the 5′ terminus, one or more nucleotides that comprise a phosphorothioate linkage; and/or
(ii) at the 3′terminus, one or more nucleotides that comprise a phosphorothioate linkage; and
(iii) a 3′-block that prevent polymerase extension on the 3′ terminus of the blocking oligonucleotide;
wherein the one or more blocking oligonucleotides bind to template sequences of non-desired library fragments, thereby blocking amplification of the non-desired library fragments by PCR.

37. The RNA-Seq based library preparation kit of aspect 36, wherein the library preparation kit further comprises:
an A-tailing mix;
an enhanced PCR mix;
a ligation mix;
a resuspension buffer;
a stop ligation buffer;
an Elute, Prime, Fragment High Concentration Mix;
a First strand Synthesis Act D Mix;
a reverse transcriptase; and
a second strand master mix.

38. The RNA-Seq based library preparation kit of aspect 37, wherein the one or more of the blocking oligonucleotides are from 15 nt to 100 nt in length, preferably wherein the blocking nucleotides are from 15 nt to 80 nt, 15 nt to 70 nt, 15 nt to 60 nt, 15 nt to 50 nt, 15 nt to 40 nt, 15 nt to 30 nt, 17 nt to 30 nt, or 20 nt to 30 nt in length.

39. An RNA-Seq based library preparation kit comprising a pool of blocking oligonucleotides, wherein a portion of the pool of blocking oligonucleotides bind to each strand of a template sequence of a non-desired fragment in a nonoverlapping and adjacent manner, thereby blocking amplification of the non-desired library fragments by PCR.

40. The RNA-Seq based library preparation kit of aspect 39, wherein the library preparation kit further comprises:
an A-tailing mix;
an enhanced PCR mix;
a ligation mix;
a resuspension buffer;
a stop ligation buffer;
an Elute, Prime, Fragment High Concentration Mix;
a First strand Synthesis Act D Mix;
a reverse transcriptase; and
a second strand master mix.

41. The RNA-Seq based library preparation kit of aspect 39 or aspect 40, wherein the pool of the blocking oligonucleotides are from 15 nt to 100 nt in length, preferably wherein the blocking nucleotides are from 15 nt to 80 nt, 15 nt to 70 nt, 15 nt to 60 nt, 15 nt to 50 nt, 15 nt to 40 nt, 15 nt to 30 nt, 17 nt to 30 nt, or 20 nt to 30 nt in length.

42. The RNA-Seq based library preparation kit of any one of aspects 39 to 41, wherein the pool of blocking oligonucleotides comprise (i) and/or (ii), and (iii):
(i) at the 5′ terminus, one or more nucleotides that comprise a phosphorothioate linkage; and/or
(ii) at the 3′terminus, one or more nucleotides that comprise a phosphorothioate linkage; and
(iii) a 3′-block that prevent polymerase extension on the 3′ terminus of the blocking oligonucleotide.

43. The RNA-Seq based library preparation kit of aspect 42, wherein the 3′-block is selected from a C₃-spacer, 3′ inverted bases, 3′ phosphorylation, 3′ dideoxy bases or 3′ non-complementary overhanging bases.

A number of embodiments of the disclosure have been described. Nevertheless, it will be understood that various modifications may be made without departing from the spirit and scope of the disclosure. Accordingly, other embodiments are within the scope of the following claims.

1. A method to selectively deplete non-desirable fragments from amplified DNA or cDNA libraries by using one or more blocking oligonucleotides, comprising:
amplifying in a polymerase chain reaction (PCR), a plurality of library fragments comprising a double stranded template sequence including adapter sequences, wherein a portion of the fragments comprise non-desirable fragments that are not to be analyzed;
wherein the PCR reaction comprises a plurality of fragments, a polymerase, dNTPS, PCR primers, and one or more blocking oligonucleotides, wherein the one or more blocking oligonucleotides comprise (i) and/or (ii), and (iii):
(i) at the 5′ terminus, one or more nucleotides that comprise a phosphorothioate linkage; and/or
(ii) at the 3′terminus, one or more nucleotides that comprise a phosphorothioate linkage; and
(iii) a 3′-block that prevent polymerase extension on the 3′ terminus of the blocking oligonucleotide;
wherein the one or more blocking primers bind to the template sequences of non-desired fragments, thereby blocking amplification of the non-desired fragments by PCR.

2. The method of claim 1, wherein the one or more of the blocking oligonucleotides are from 15 nt to 100 nt in length.

3. The method of claim 1, wherein when the polymerase has 5′ to 3′ exonuclease activity, then the one or more of the blocking oligonucleotides comprise at the 5′ terminus, 1 to 5 nucleotides that comprise a phosphorothioate linkage.

4. The method of claim 1, wherein when the polymerase has 3′ to 5′ proofreading activity, then the one or more of the blocking oligonucleotides comprise at the 3′ terminus, 1 to 5 nucleotides that comprise a phosphorothioate linkage.

5. (canceled)

6. The method of claim 1, wherein the 3′-block is selected from a C₃-spacer, 3′ inverted bases, 3′ phosphorylation, 3′ dideoxy bases or 3′ non-complementary overhanging bases.

7. The method of claim 1, wherein the amplified libraries comprise template sequences from cDNA or from DNA.

8. (canceled)

9. The method of claim 1, wherein the adapter sequences are from Y-shaped adapters that have been ligated to each end of a template sequence.

10. The method of claim 1, wherein the one or more blocking oligonucleotides bind to template sequences from mtDNA, rRNAs and/or globin.

11. The method of claim 10, wherein the one or more blocking oligonucleotides comprise a pool of blocking oligonucleotides that bind to template sequences from 18S rRNA, 5.8S rRNA, and/or 28S RNA.

12. (canceled)

13. The method of claim 1, wherein the amplified DNA or cDNA libraries are analyzed by using next generation sequencing.

14. The method of claim 1, wherein the PCR amplification step is preceded by the following steps:
obtaining an RNA sample;
fragmenting the RNA;
reverse transcribing the RNA fragments to cDNA;
blunt ending the cDNA and adding an A nucleotide to the 3′ end of the blunt ended cDNA; and
ligating the A-tailed cDNA with adapters comprising a non-complemented T nucleotide at the 3′ end.

15. The method of claim 14, wherein prior to reverse transcribing the RNA fragments to cDNA, the RNA sample is treated to deplete rRNA sequences from the RNA sample.

16. A method to selectively deplete non-desirable fragments from amplified DNA or cDNA libraries by using one or more blocking oligonucleotides, comprising:
amplifying in a polymerase chain reaction (PCR) reaction, a plurality of library fragments comprising a double stranded template sequence including adapter sequences, wherein a portion of the fragments comprise non-desirable fragments that contain template sequences that are not to be analyzed;
wherein the PCR reaction comprises a plurality of fragments, a polymerase, dNTPS, PCR primers, and a pool of blocking oligonucleotides, wherein a portion of the pool of the blocking oligonucleotides bind to each strand of a template sequence of a non-desired fragment;
wherein the one or more blocking primers bind to the template sequences of non-desired fragments, thereby blocking amplification of the non-desired fragments by PCR.

17. The method of claim 16, wherein the pool of blocking oligonucleotides are from 15 nt to 100 nt in length.

18. The method of claim 16, wherein the pool of blocking oligonucleotides comprise blocking oligonucleotides which bind to the strands of the template in a nonoverlapping and adjacent manner.

19. The method of claim 18, wherein the pool of blocking oligonucleotides comprise blocking oligonucleotides that are reverse-complement to other blocking oligonucleotides.

20. The method of claim 16, wherein the pool of blocking oligonucleotides comprise (i) and/or (ii), and (iii):
(i) at the 5′ terminus, one or more nucleotides that comprise a phosphorothioate linkage; and/or
(ii) at the 3′terminus, one or more nucleotides that comprise a phosphorothioate linkage; and
(iii) a 3′-block that prevent polymerase extension on the 3′ terminus of the blocking oligonucleotide.

21. The method of claim 20, wherein when the polymerase has 5′ to 3′ exonuclease activity, then the one or more of the blocking oligonucleotides comprise at the 5′ terminus, 1 to 5 nucleotides that comprise a phosphorothioate linkage.

22. The method of claim 20, wherein when the polymerase has 3′ to 5′ proofreading activity, then the one or more of the blocking oligonucleotides comprise at the 3′ terminus, 1 to 5 nucleotides that comprise a phosphorothioate linkage.

23. (canceled)

24. The method of claim 20, wherein the 3′-block is selected from a C₃-spacer, 3′ inverted bases, 3′ phosphorylation, 3′ dideoxy bases or 3′ non-complementary overhanging bases.

25. The method of claim 16, wherein the amplified libraries comprise template sequences from cDNA or gDNA.

26. (canceled)

27. The method of claim 16, wherein the adapter sequences are from Y-shaped adapters that have been ligated to each end of a template sequence.

28. The method of claim 16, wherein the pool of blocking oligonucleotides bind to template sequences from mtDNA, rRNAs and/or globin.

29. The method of claim 16, wherein the pool of blocking oligonucleotides bind to template sequences from 18S rRNA, 5.8S rRNA, and/or 28S RNA.

30. (canceled)

31. The method of claim 16, wherein the amplified DNA or cDNA libraries are analyzed by using next generation sequencing.

32. The method of claim 16, wherein the PCR amplification step is preceded by the following steps:
obtaining an RNA sample;
fragmenting the RNA;
reverse transcribing the RNA fragments to cDNA;
blunt ending the cDNA and adding an A nucleotide to the 3′ end of the blunt ended cDNA; and
ligating the A-tailed cDNA with adapters comprising a non-complemented T nucleotide at the 3′ end.

33. The method of claim 32, wherein prior to reverse transcribing the RNA fragments to cDNA, the RNA sample is treated to deplete rRNA sequences from the RNA sample.

34. An RNA-Seq based library preparation kit comprising one or more blocking oligonucleotides, wherein the one or more blocking oligonucleotides comprise (i) and/or (ii), and (iii):
(i) at the 5′ terminus, one or more nucleotides that comprise a phosphorothioate linkage; and/or
(ii) at the 3′terminus, one or more nucleotides that comprise a phosphorothioate linkage; and
(iii) a 3′-block that prevent polymerase extension on the 3′ terminus of the blocking oligonucleotide;
wherein the one or more blocking oligonucleotides bind to template sequences of non-desired library fragments, thereby blocking amplification of the non-desired library fragments by PCR.

35. The RNA-Seq based library preparation kit of claim 34, wherein the library preparation kit further comprises:
an A-tailing mix;
an enhanced PCR mix;
a ligation mix;
a resuspension buffer;
a stop ligation buffer;
an Elute, Prime, Fragment High Concentration Mix;
a First strand Synthesis Act D Mix;
a reverse transcriptase; and
a second strand master mix.

36. The RNA-Seq based library preparation kit of claim 34, wherein the one or more of the blocking oligonucleotides are from 15 nt to 100 nt in length.

37. An RNA-Seq based library preparation kit comprising a pool of blocking oligonucleotides, wherein a portion of the pool of blocking oligonucleotides bind to each strand of a template sequence of a non-desired fragment in a nonoverlapping and adjacent manner, thereby blocking amplification of the non-desired library fragments by PCR.

38. The RNA-Seq based library preparation kit of claim 37, wherein the library preparation kit further comprises:
an A-tailing mix;
an enhanced PCR mix;
a ligation mix;
a resuspension buffer;
a stop ligation buffer;
an Elute, Prime, Fragment High Concentration Mix;
a First strand Synthesis Act D Mix;
a reverse transcriptase; and
a second strand master mix.

39. The RNA-Seq based library preparation kit of claim 37, wherein the pool of the blocking oligonucleotides are from 15 nt to 100 nt in length.

40. The RNA-Seq based library preparation kit of claim 37, wherein the pool of blocking oligonucleotides comprise (i) and/or (ii), and (iii):
(i) at the 5′ terminus, one or more nucleotides that comprise a phosphorothioate linkage; and/or
(ii) at the 3′terminus, one or more nucleotides that comprise a phosphorothioate linkage; and
(iii) a 3′-block that prevent polymerase extension on the 3′ terminus of the blocking oligonucleotide.

41. The RNA-Seq based library preparation kit of claim 40, wherein the 3′-block is selected from a C₃-spacer, 3′ inverted bases, 3′ phosphorylation, 3′ dideoxy bases or 3′ non-complementary overhanging bases.
